(19) United States
(12) Patent Application Publication
(10) Pub. No.: US 2020/0261023 A1
(43) Pub. Date: Aug. 20, 2020

(54) Ascertaining, Reporting, and Influencing Physical Attributes And Performance Factors of Athletes

(71) Applicant: Athletai Co., Davie, FL (US)

(72) Inventors: Jacob Werbin, Davie, FL (US); Carlos Liverani, Weston, FL (US); Zoheb Anjum, Miami, FL (US)

(21) Appl. No.: 16/788,366

(22) Filed: Feb. 12, 2020

Related U.S. Application Data
(60) Provisional application No. 62/805,911, filed on Feb. 14, 2019.

(51) Int. Cl. A61B 5/00; A61B 5/0492; A61B 5/11; A61B 5/04; A61B 5/22; A61B 5/01; A41D 13/00; A41D 1/00

(52) U.S. Cl. CPC A61B 5/6804; A61B 5/0492; A61B 5/1107; A61B 5/04012; A61B 5/224; A61B 5/01; A41H 3/04; A41D 1/002; A61B 2562/0219; A61B 2562/0247; A61B 2503/10; A61B 2562/0209; A41D 13/0002

(57) ABSTRACT

Athlete technology including athlete garments, athlete sensors, athlete data processors, athlete data networks, and other features, functions, and components.

### CLAIM OF PRIORITY

This application claims priority under 35 U.S.C. 119(e) to U.S. Provisional Application No. 62/805,911, filed on Feb. 14, 2019. The content of this application is incorporated here by reference in its entirety.

### TECHNICAL FIELD

This description relates to ascertaining, reporting, and influencing physical attributes and performance attributes of athletes.

### BACKGROUND

Individual athletes, groups of athletes, and teams of athletes can benefit from efforts to ascertain, report, and influence their static and dynamic physical attributes and performance factors. Other people and institutions associated with the athletes, groups of athletes, and teams of athletes can also benefit from such efforts. These other people and institutions can include family members, friends, team mates, coaches, trainers, educational institutions that host teams, gamblers, fans, physicians and other medical care providers, and observers, to name a few. We use the term “athlete stakeholders” broadly to include, for example, athletes, groups of athletes, teams of athletes, and such other people and institutions.

We use the term “athlete” broadly to include, for example, not only people who, in the conventional sense, engage in organized, directed, or sustained sporting enterprises, but also anyone who engages in physical activity as part of a job, profession, an application, or recreational endeavor, among others.

We sometimes use the term “athlete participants” broadly to include, for example, individual athletes, groups of athletes, and teams of athletes, in particular, those who use, subscribe to, engage with, or participate in one or more features of the technology described here.

We use the term “athlete physical attributes” of an athlete broadly to include, for example, any quality, characteristic, state, condition, aspect, nature, or trait of or associated with the athlete's body or an internal or external part or parts of the athlete's body. Athlete physical attributes can include static attributes, such as size, shape, structure, orientation, temperature, wetness, resilience, hardness, electrical properties, and other qualities at one or more specific times. Athlete physical attributes can include dynamic attributes such as changing static attributes, for example, speed, acceleration, motion, combinations of motions, and others, and combinations of them. Athlete physical attributes can include attributes of body systems of the athlete's body, for example, the cardiorespiratory system or the musculoskeletal system. Some athlete physical attributes can be measured and quantified directly, such as weight, height, running speed, and others. Some athlete physical attributes can be expressed more subjectively, such as strength, fitness, and agility. Sometimes some or all of the athlete physical attributes can be referred to as the athlete's physiology.

Athletes use their athlete physical attributes when they engage in athlete activity. Athlete activity typically involves athlete motion including gross motion of the athlete's entire body from one location to another and particular motion of one or more athlete body parts or athlete body segments of the athlete's body (or combinations of them). For example, the athlete could be running to a spot on a basketball court or lifting an arm. We use the term “athlete activity” broadly to include, for example, exercising, working out, training, practicing, rehabilitating, competing, being coached, engaging in team sports, and others, and combinations of them. Athlete activities can be engaged in individually, in groups, or in teams, for example.

We use the term “athlete performance factors” broadly to include, for example, success, failure, winning, losing, improvement, decline, injury, risk of injury, fatigue, physiology, psychology, and other factors, degrees of any of those, changes in any of those, and combinations of any of those, in particular with respect to athlete activity.

Athlete participants and athlete stakeholders are typically interested in physical attributes of the athlete participants, athlete activities of the athlete participants, and athlete performance factors of the athlete participants. Athlete participants and athlete stakeholders are especially attentive to improving athlete performance factors and athlete physical attributes.

Athlete physical attributes, athlete activities, and athlete performance factors are closely related to two main body systems: the cardiorespiratory system and the musculoskeletal system.

The cardiorespiratory system includes the heart, the lungs, and the vasculature (which we sometimes call cardiorespiratory parts). Measurement techniques typically used to characterize the cardiorespiratory system include thoracic electrical bio-impedance, electrocardiography, and near infrared spectroscopy.

The musculoskeletal system includes the bones, ligaments, tendons, and muscles (which we sometimes call musculoskeletal parts). Measurement techniques typically used to characterize the musculoskeletal system include surface electromyography, force myography, and inertial measurement units.

The effectiveness of the cardiorespiratory system and the effectiveness of the musculoskeletal system of a given athlete and of different athletes can differ over time and depending on the context. For example, a long-distance runner may have a highly efficient cardiorespiratory system supplying a musculoskeletal system of average efficiency, or an average cardiorespiratory system supplying a highly efficient musculoskeletal system, or, if the person is an elite athlete, both of the person's systems may be highly efficient.

The cardiorespiratory system and the musculoskeletal system work together through the intermediary of the nervous system and in particular the brain. Among other things, the cardiorespiratory system provides the musculoskeletal system with oxygen and helps carry away by-products like lactic acid.

The cardiorespiratory system, the musculoskeletal system, and the nervous system work together in what can be considered an athlete motion control system. The main components of a typical control system are sensors and actuators and a feedback path from the sensors to the actuators. An actuator is typically a component that moves or otherwise actuates something. An actuator typically requires one or more input signals to cause it to actuate. The input signals are typically received through the feedback path from the sensors. A sensor is typically a component that measures, detects, or is otherwise aware of a condition or state of something. A sensor provides an output that is delivered to the feedback path for use in providing an input signal to the actuators.

The athlete motion control system includes many sensors that are constantly and automatically involved in perception of physical attributes of one or more parts of the body in a process called proprioception. Any physiological structure (e.g., sensor) of the body that contributes to proprioception is called a proprioceptor, an example being muscle spindles that sense a muscle's contraction velocity. Using signals provided by the proprioceptors, the brain can understand where the body is in space and how the parts or segments of the musculoskeletal system of the body are positioned in relation to one another. Based on that understanding, the brain can issue signals to musculoskeletal parts or segments to control their position, orientation, or motion in one or more intended ways for one or more intended purposes. Physical attributes of the musculoskeletal parts or segments are in turn sensed by proprioceptors as part of the continuously operating feedback loop of the athlete motion control system.

The effectiveness of athletes' athlete motion control systems as reflected in athlete performance factors of the athletes vary. The effectiveness of a particular athlete's athlete motion control system may be limited based on, for example, genetics, injury, health, training, experience, physiology, psychology, other factors, and combinations of them. The effectiveness of a particular athlete's athlete motion control system can be improved by engaging in athlete activities, including training, coaching, and rehabilitation, and by the athlete being aware of and sensitive to athlete physical attributes, athlete activity, and athlete performance factors, including for example effects of training techniques, awareness of fatigue, and reduction of injury.

Here, we describe athlete technology for, among other things, acquiring and reporting athlete performance information such as athlete data and athlete insights about athlete physical attributes (e.g., the athlete motion control system) and athlete performance factors of one or more athlete participants, and for influencing athlete physical attributes, athlete performance factors, and athlete activities.

We use the term “athlete data” broadly to include, for example, any quantitative or qualitative information about athlete physical attributes, athlete performance factors, and athlete activities. Athlete data can apply to moments in time and to time periods. Athlete data can include raw data, such as signals from sensors, and processed data derived, for example from raw data.

We use the term “athlete insights” broadly to include, for example, human or machine interpretations, inferences, predictions, or conclusions, among other things, about athlete physical attributes, athlete performance factors, and athlete activities. Athlete insights can be expressed quantitatively, in prose, visually, and in a variety of other ways and combinations of them. Athlete insights can be generated by human beings or by machine such as by machine learning systems.

We use the term “athlete technology” broadly to include, for example, any device or technique useful to and used by athlete participants and athlete stakeholders for, among other things, ascertaining, reporting, and influencing athlete activities, athlete performance factors, and athlete physical attributes. Athlete technology can include, for example, hardware and software devices such as sensors, hubs, networks, mobile phones, tablets, computers, servers, cloud platforms, user interfaces, garments, and portions of garments, to name a few. Athlete technology can include, for example, methods, processes, techniques, and approaches for ascertaining, reporting, and influencing athlete activities, athlete performance factors, and athlete physical attributes, among other things. Athlete technology can include and implement feedback loops encompassing sensors, computational facilities, and user interfaces. In some cases, the feedback loops can assist and work in conjunction with the athlete motion control system to assist the brain of the athlete participant in a way that enables the athlete participant to understand, alter, or improve athlete activity, athlete physical attributes, or athlete performance factors. For example, by applying the athlete technology to athlete activity of a basketball player, the basketball player may be able to jump higher for rebounds while reducing the chance of injury.

The athlete technology that we describe here enables athlete participants and athlete stakeholders to favorably influence athlete physical attributes, athlete activities, and athlete performance factors. For this purpose, the athlete technology can be implemented by one or more devices, techniques, and concepts, and combinations of them, including the ones discussed below.

Physical attributes of athlete participants are measured by athlete sensors of various kinds and operated in various modes to acquire rich, complete, and accurate athlete data. In some phases of the use of the athlete technology, the athlete physical attributes of athlete participants are in effect calibrated by applying athlete sensors including athlete sensors held on calibration garments and by video cameras that observe the athlete participants. The calibration athlete data is processed to derive information about the athlete physical attributes and to provide inputs for developing and training predictive models customized for the respective athlete participants and optimizing the placement of sensors (for example, on athlete garments) with respect to the body of the athlete participant. The athlete technology includes customized athlete garments that have configurations and sizes and bear sensors at locations that are optimum for acquisition of athlete data during athlete activity.

When the customized athlete garments are worn during athlete activity, large bodies of complete, accurate, and detailed athlete data associated with the athlete activity, athlete physical attributes, and athlete performance factors of corresponding athlete participants can be acquired. The acquired athlete data is processed and interpreted and used for a wide variety of purposes in the athlete technology. Among other things, the acquired athlete data can be used to train machine learning models of the athlete physical attributes and athlete performance factors of the athlete participants. The models can be associated with specific individual athlete participants or with groups of athlete participants or teams of athlete participants. During athlete activity, the athlete models can predict athlete performance factors, such as athlete fatigue, potential athlete injury, improved athlete performance, and other factors. The athlete models can be generated, maintained, and run on a variety of devices and combinations of them, including processors attached to the athlete garments, devices such as mobile phones associated with the athlete participants and the athlete stakeholders, central servers, and servers located on cloud platforms. Among other things, the athlete models can provide athlete insights.

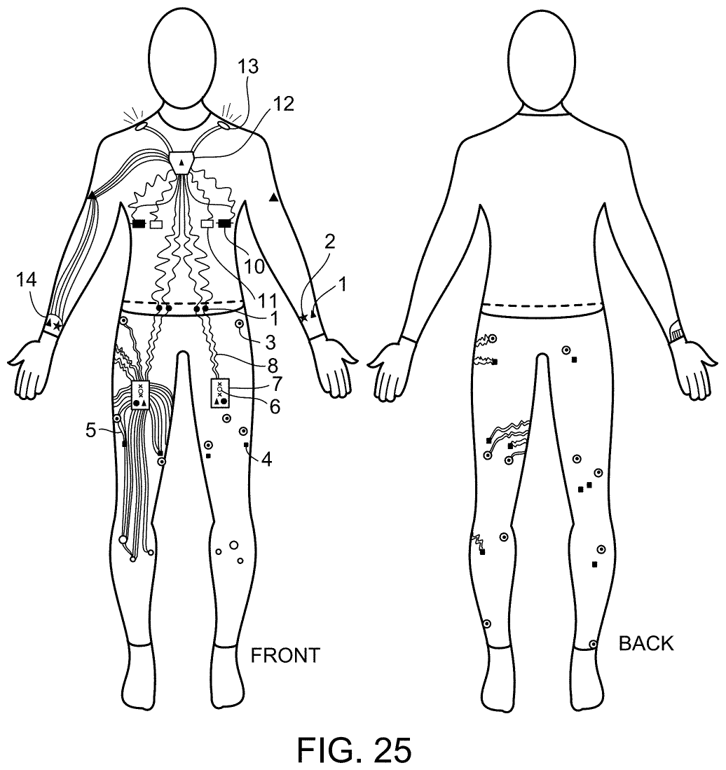

We use the term “athlete garment” broadly to include, for example, any item of clothing or apparel, part of an item of clothing or apparel, or any other device or contrivance, that can be worn by an athlete. In some implementations, an athlete garment is sized or configured to fit closely to the skin of an athlete. In some implementations, and athlete government is in the form of a compression garment. An athlete garment can be instrumented by including one or more sensors, networks, communicators, hardware, software, or electronic devices. An athlete garment need not be configured as a conventional garment, but can be designed especially for use with the athlete technology, such as an athlete garment in the form of a fabric band worn around the chest or a thigh or forearm, for example. See FIG. 25, for example.

We use the term “athlete model” broadly to include, for example, any software device designed or configured to predict, simulate, imitate, or emulate athlete activity, athlete physical attributes, and athlete performance factors based on athlete data. Athlete models can include predictive models, simulation models, machine learning models, and combinations of them.

Athlete data and athlete insights can be used for a wide variety of purposes and in a wide variety of ways. Among other things, athlete data and athlete insights can be communicated to athlete participants and athlete stakeholders through communication networks and then presented through user interfaces of devices. Athlete data and athlete insights can be aggregated, analyzed, and reported. Implementations of the athlete technology can include feedback loops that include the sensors, the athlete data, the athlete models, the athlete insights, communication networks, user devices and user interfaces, and the brains of athlete participants and trainers and coaches and other athlete stakeholders. The feedback loops can facilitate improvements in athlete activities, athlete physical attributes, and athlete performance factors. In some implementations, an athlete participant can receive immediate and continuous feedback through a user interface of a mobile device while engaging in athlete activity to assist the athlete participant in improving with respect to the athlete activity, athlete physical attributes, and athlete performance factors. The feedback can be based on and include rich, deep, complete, and accurate athlete data and (e.g., subjective) athlete insights generated by custom athlete models generated for that athlete participant.

Among other advantages of the athlete technology are that it enhances the ability of athlete participants and athlete stakeholders to work together as a group in a high-performance environment to favorably influence athlete activities, athlete physical attributes, and athlete performance factors. Such a team can include athlete participants, coaches, doctors, therapists, and, for example, team managers.

The athlete technology that we describe here can instrument, acquire athlete data, model, quantify, and provide detailed insights about the behavior, performance, health, and changes in individual muscles of an athlete participant at a given moment in time and over time. Each muscle of an athlete participant can be quantified and modeled to a high level of accuracy and precision and the resulting information can be used to improve athlete physical attributes and athlete performance factors, among other things.

### SUMMARY

In general, in an aspect, athlete data is received from one or more inertial measurement units (IMUs) associated with one or more muscles of an athlete engaging in athlete activity. Athlete data is received from one or more Laplacian electrodes associated with the one or more muscles of the athlete and indicative of sEMG signals. Athlete data is received from one or more pressure sensors indicative of force imparted by muscle heads of the one or more muscles. One or more athlete performance factors of the athlete associated with the one or more muscles are characterized based on the athlete data from the one or more inertial measurement units, from the one or more Laplacian electrodes, and from the one or more pressure sensors.

In general, in an aspect, athlete data is received including motion data from one or more inertial measurement units associated with one or more muscles of an athlete engaging in athlete activity. The motion data is used to characterize the athlete activity or a status of the athlete. Data including one or more body temperatures of the athlete is received from one or more temperature sensors integrated with the initial measurement units. One or more athlete performance factors are determined based on the one or more body temperatures of the athlete.

In general, in an aspect, athlete data is received from concentric electrodes in contact with skin of an athlete at one or more innervation zones of a muscle group. Electromyography information is determined about the muscle group based on signal morphology of the received athlete data.

In general, in an aspect, an athlete garment includes a fabric, conductive elements incorporated into the fabric by weaving, printing, or depositing in a concentric pattern or stitching, gluing, or adhering preformed conductive elements to provide electrodes to make contact with skin of an athlete when the athlete garment is worn. Insulated conductive elements are incorporated into the fabric to carry signals from the conductive elements to analog circuitry.

In general, in an aspect, athlete data is received from force sensors situated at muscle heads of muscles of an athlete. Athlete data is received from electromyography sensors at one or more innervation zones of the muscles of the athlete. Based on the athlete data from the force sensors and from the electromyography sensors, electromechanical delays are determined between the appearance of recruitment signals in the muscles and the generation of forces at the muscle heads.

In general, in an aspect, the locations of innervation zones of one or more muscles of an athlete are determined algorithmically. A custom athlete garment is formed for the athlete. The custom athlete garment bears electromyography sensors at locations corresponding to the locations of the innervation zones.

In general, in an aspect, athlete data is received from electromyography sensors at one or more innervation zones of one or more muscles of an athlete. Athlete data is received from force myography sensors at one or more muscle heads of the muscles of the athlete. A degree of fatigue of the athlete is determined based on a comparison of the athlete data from the force myography sensors and the athlete data from the electromyography sensors.

In general, in an aspect, athlete data is received from electromyography sensors located at one or more innervation zones of one or more muscles of an athlete. Power spectral shifts in the athlete data from the electromyography sensors are analyzed. Athlete physical attributes or athlete performance factors are determined based on the analyzed spectral shifts.

In general, in an aspect, athlete data is received from force myography sensors located at one or more muscle heads of one or more muscles of an athlete. Based on the athlete data from the force myography sensors information is determined about the lengthening of one or more of the muscles. Information is inferred about flexibility or overextension of the one or more muscles based on the information about the lengthening.

In general, in an aspect, athlete data is received from sensors associated with a muscle of an athlete, the athlete data indicating electrical activity at innervation zones of the muscle and motion at a muscle head of the muscle. Based on the athlete data, behavior of the muscle is quantified and modeled accurately and precisely. The result of the quantification modeling is used to improve athlete physical attributes and athlete performance factors.

In general, in an aspect, athlete data is sensed from sensors located on an athlete garment worn by an athlete during an athlete activity. Compressed sensing techniques are applied to the sensing of the athlete data to reduce the volume and rate of athlete data being sensed. The compressively sensed athlete data is wirelessly sent to a processing device not located on the athlete garment.

In general, in an aspect, sensors are used to determine locations of innervation zones of muscles of each of two or more different athletes of a team of athletes. A custom sized and shaped athlete garment is formed for each of the respective athletes. Sensors are incorporated in each of the athlete garments sensors at locations based on the determined locations of the innervation zones of the muscles of the corresponding athlete.

In general, in an aspect, electronic devices are used to determine information about a shape of the body of an athlete, locations of joints of the athlete, axes of joints of the athlete, the stretching and range of motion of respective parts of the body of the athlete, and the locations of innervation zones of muscles of the athlete. A custom athlete garment is formed for the athlete incorporating sensors at locations determined based on the information determined using the electronic devices.

In general, in an aspect, a measurement frame is established. The measurement frame is marked on an athlete prior to an athlete activity. A high definition sEMG mat is applied on a muscle of interest of the athlete, the location of the mat on the body of the athlete is marked to provide a reference frame for attribution of athlete data generated by the mat during the athlete activity. A photogrammetry process or a laser scan is applied to the athlete during the athlete activity.

In general, in an aspect, information is captured about two or more different poses of an athlete. Parts of the body of the athlete that are subject to changes in shape or size between two or more of the different poses are determined. Based on the determination of the parts of the body subject to such changes, panels of a custom athlete garment are determined to which to impart additional give to reduce boundary shear locations at which fabric parts to be assembled to form the custom athlete garment are stitched. The custom athlete garment is formed from the panels. The custom athlete garment includes a custom compression athlete garment.

In general, in an aspect, locations are determined on the body of an athlete where motion of the athlete during athlete activity causes skin stretching and the amounts of skin stretching caused. Patterns are determined for conductive elements configured to carry signals from sensors to be placed at locations adjacent the skin of the athlete to processing circuitry located remotely from the sensors. An athlete garment is formed for the athlete by integrating into the athlete garment conductive elements that match paths determined to be associated with relatively smaller amounts of skin stretching.

In general, in an aspect, an athlete garment includes garment fabric configured to fit an athlete during athlete activity. Sensors are incorporated into the garment fabric at predetermined locations associated with innervation zones and muscle heads of muscles of the athlete. A user interface displays configured to receive and display information about athlete physical attributes or athlete performance factors or both as immediate feedback to the athlete during athlete activity. The user interface displays attached to the athlete garment in a manner to be visible at a middle forearm of the athlete. In some implementations, small sound sources are included in the athlete garment.

In general, in an aspect, an apparatus includes a custom athlete garment configured to fit a particular athlete. Sensors are integrated in the custom athlete garment and positioned with respect to muscle heads and innervation zones of muscles or other anatomical landmarks, such as TEB electrodes at the sixth intercostal space or the xiphoid process of the athlete to generate athlete data during athlete activity. Electronics couple two or more of the sensors separately to a wireless communication network to deliver athlete data from the respective sensors to a processor for analysis.

In general, in an aspect, an apparatus includes a custom athlete garment configured to fit a particular athlete. Sensors are integrated in the custom athlete garment and positioned relative to locations on the skin of the athlete to generate athlete data of at least two of the following kinds: electromyography, force myography, temperature, humidity, SmO₂, heart rate, and heart rate variability. In some implementations, signals athlete data generated by force myography sensors can be used to derive mechanomyograms indicative of vibrations of muscles, spectral shifts, and information relating to fatigue.

In general, in an aspect, sensors are integrated in a custom athlete garment. RR intervals are detected in the heart signal of an athlete during athlete activity. Statistical measures of the RR intervals are determined as indicators of athlete physical attributes or athlete performance factors or both. The statistical measures include at least one of: skewness of successive RR intervals, difference between the median RR interval and the mean RR interval.

In general, in an aspect, sensors are integrated in a custom athlete garment. RR intervals are detected in the heart signal of an athlete during athlete activity. An ability of the athlete to restore normal heart rate variability after the athlete activity is compared to an ability of the athlete to restore normal heart rate variability after previous athlete activity or to an ability of other athletes to restore normal heart rate variability after athlete activity.

In general, in an aspect, an apparatus includes a custom athlete garment configured to fit a particular athlete. A near infrared spectroscopy sensor is integrated into fabric of the custom athlete garment in a position selected to face skin of the particular athlete at a location effective for near infrared spectroscopy.

In general, in an aspect, athlete data is received from sensors integrated in a custom athlete garment of an athlete engaged in an athlete activity. The athlete data includes muscle hemodynamics, hydration, heart rate, muscle activation, and motion. The athlete data is used to determine kinesiology or physiology or both of the athlete in real time as the athlete engages in the athlete activity. In some implementations, the athlete data is associated with the occurrence of an injury and can be used for at least one of the following activities: understanding the mechanics and physiological context of the injury, improving recovery from the injury, adjusting athlete models, reducing chances of injuries for other athlete participants, and others.

In general, in an aspect, athlete data is received from sensors integrated in a custom athlete garment of an athlete engaged in an athlete activity. At a processor integrated in the custom athlete garment or at a location off the athlete garment, a recurrent neural network process is applied to the received athletic data. The result of applying the recurrent neural network result is used in a cloud server for decompression, de-encoding, and processing. In some implementations, a processor integrated in the custom athlete garment can comprise a neuromorphic processor applying compressed learning.

In general, in an aspect, athlete data is received from sensors integrated in a custom athlete garment of an athlete engaged in an athlete activity. Machine learning algorithms are applied to the received athlete data to generate insights into athlete physical attributes and athlete performance factors of the athlete engaged in the athlete activity. The insights are communicated to athlete stakeholders and to the athlete engaged in the athlete activity.

These and other aspects, features, implementations, and advantages (1) can be expressed as methods, apparatus, systems, components, program products, business methods, means or steps for performing functions, and in other ways, and (b) will become apparent from the following description and from the claims.

### DESCRIPTION

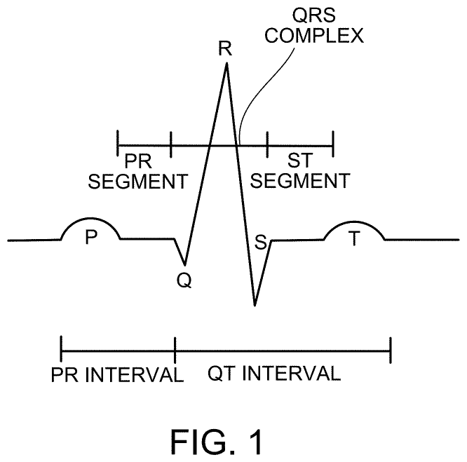

FIG. 1 is a timing diagram.

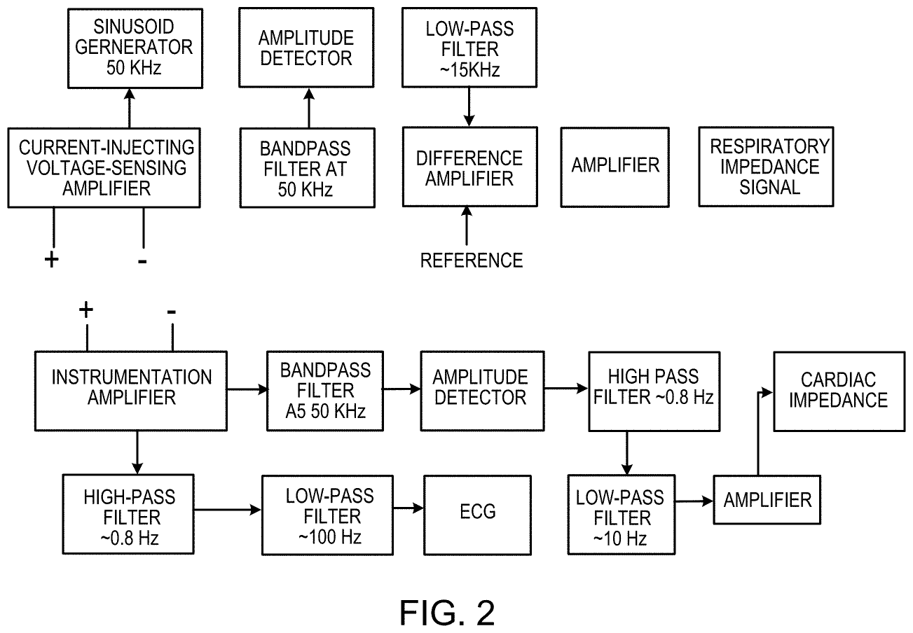

FIGS. 2-4, and 6 through 8 are block diagrams.

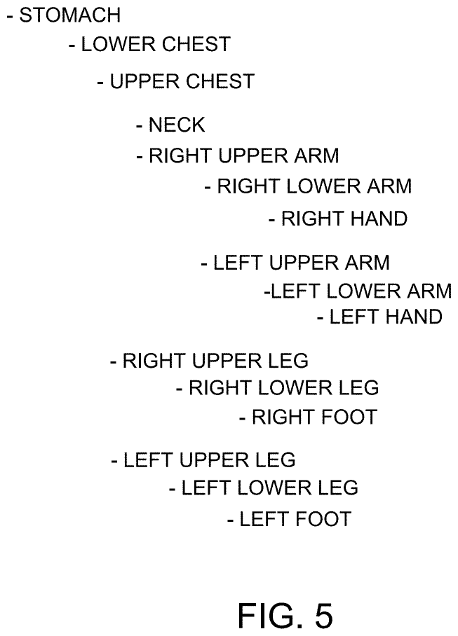

FIG. 5 is a hierarchy chart.

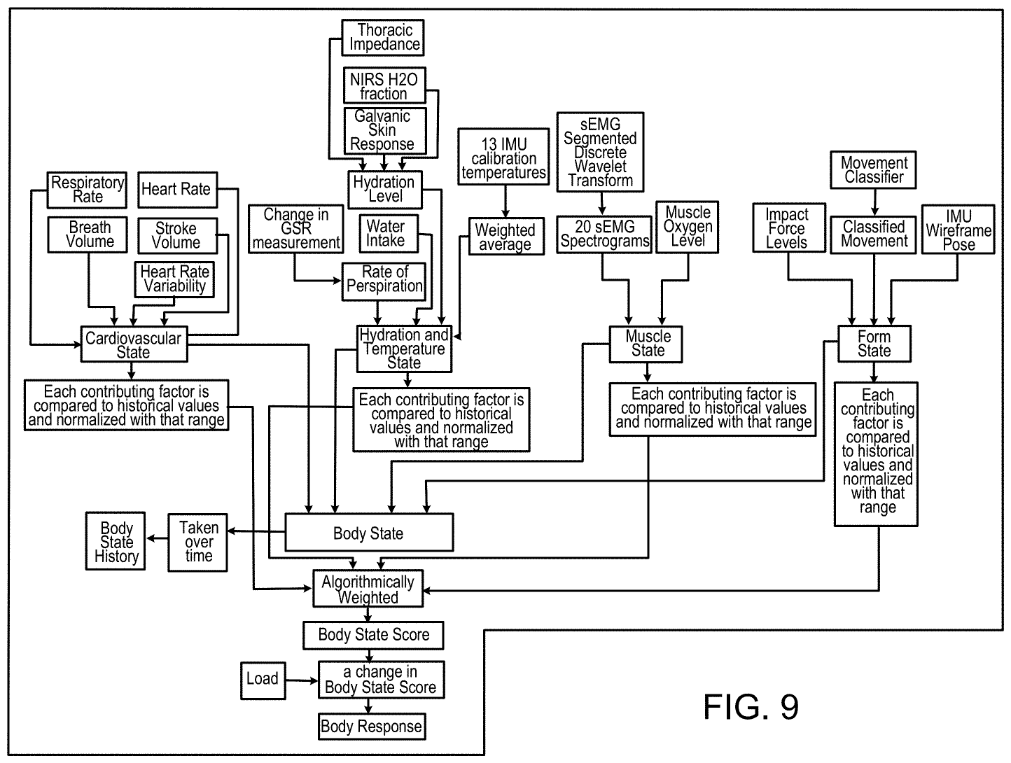

FIG. 9 shows an overall functional and structural block diagram of the athlete technology incorporating features described above and others.

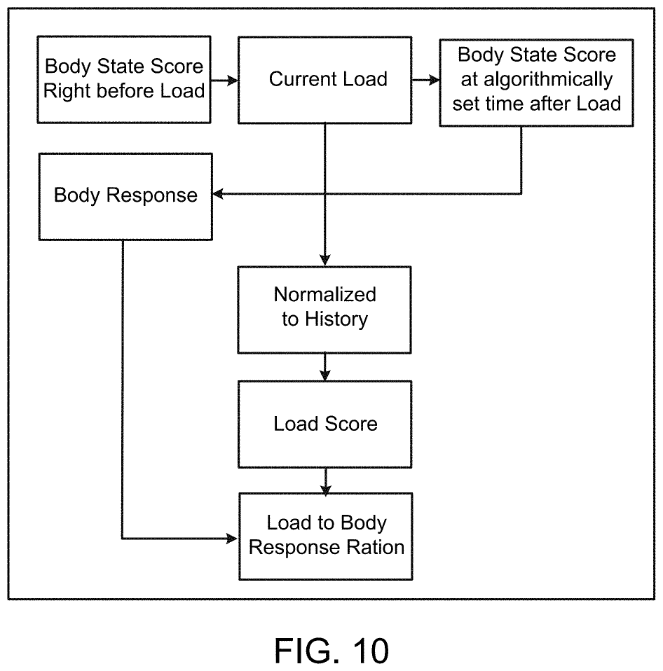

FIG. 10 shows a block diagram of a process for determining load to body response ratio in the athlete technology.

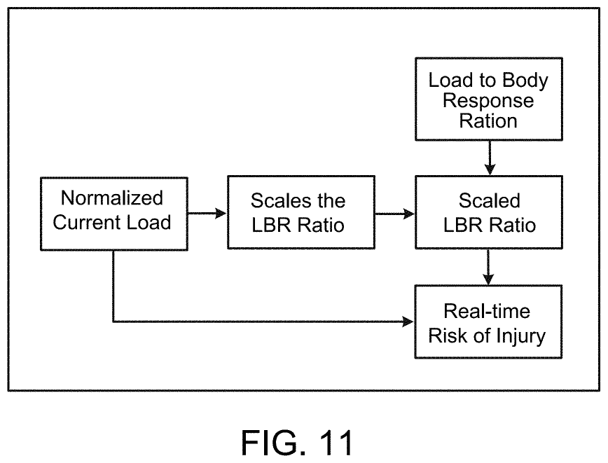

FIG. 11 shows a block diagram of a process for determining real-time risk of injury based on a load to body response ratio in the athlete technology.

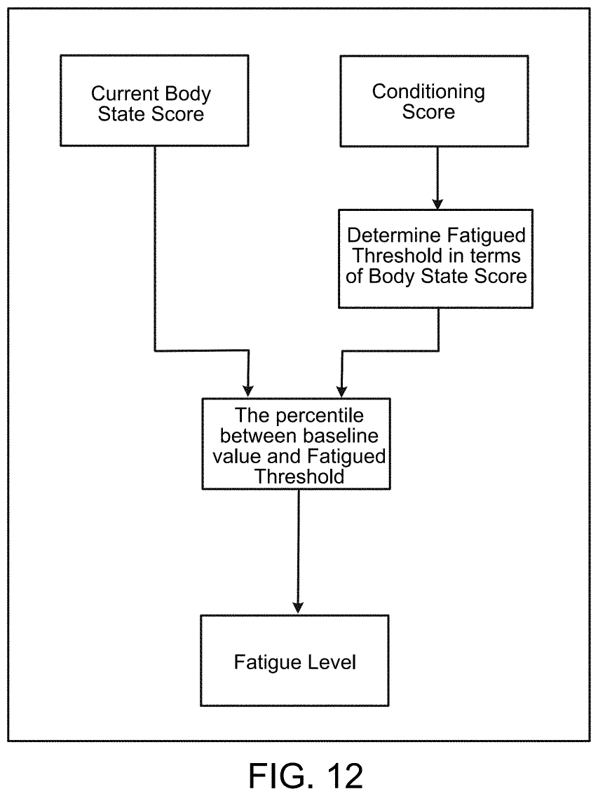

FIG. 12 shows a block diagram of a process for determining fatigue level based on a current body state score and a conditioning score in the athlete technology.

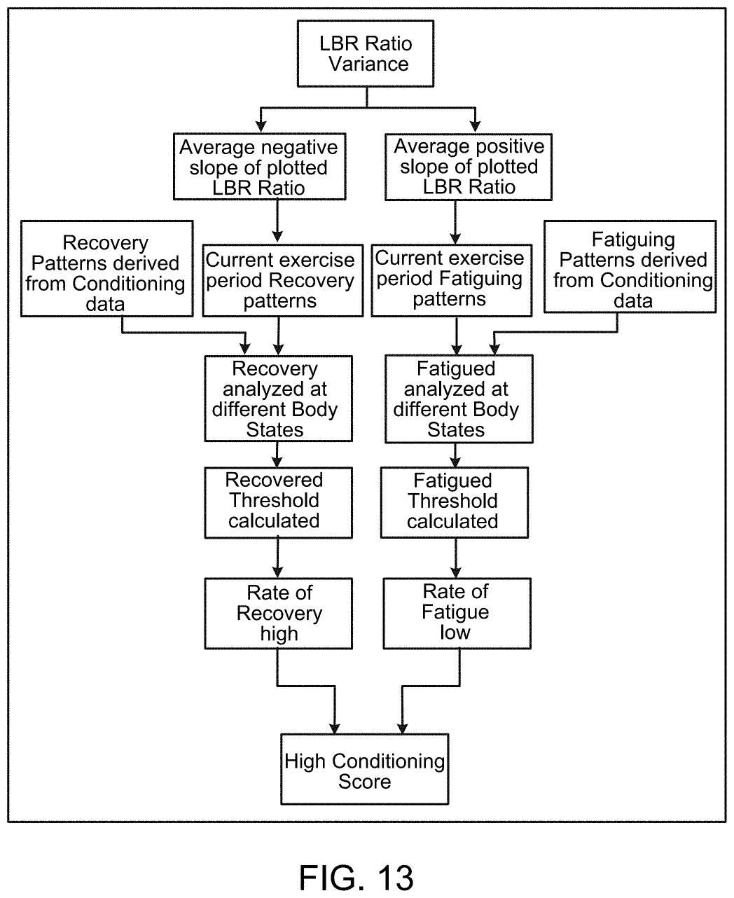

FIG. 13 shows a block diagram of a process for determining a high conditioning score.

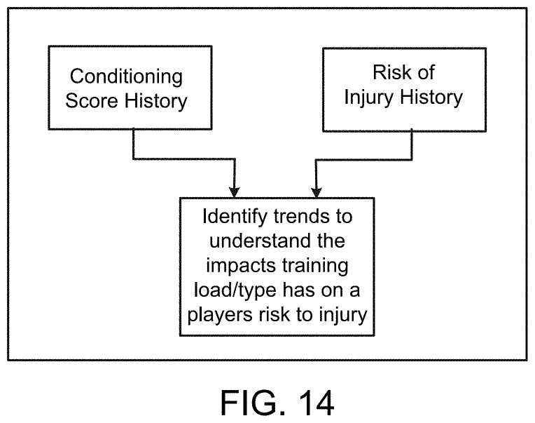

FIG. 14 shows a block diagram of a process for identifying trends to understand the impacts training load/type has on a players risk to injury.

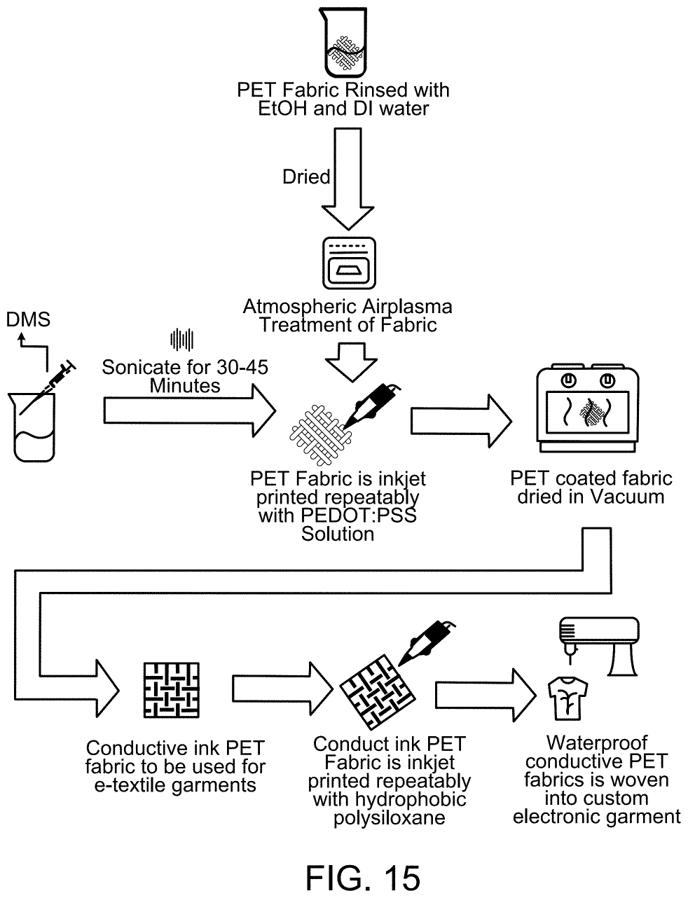

FIG. 15 shows a flow diagram of a fabrication process.

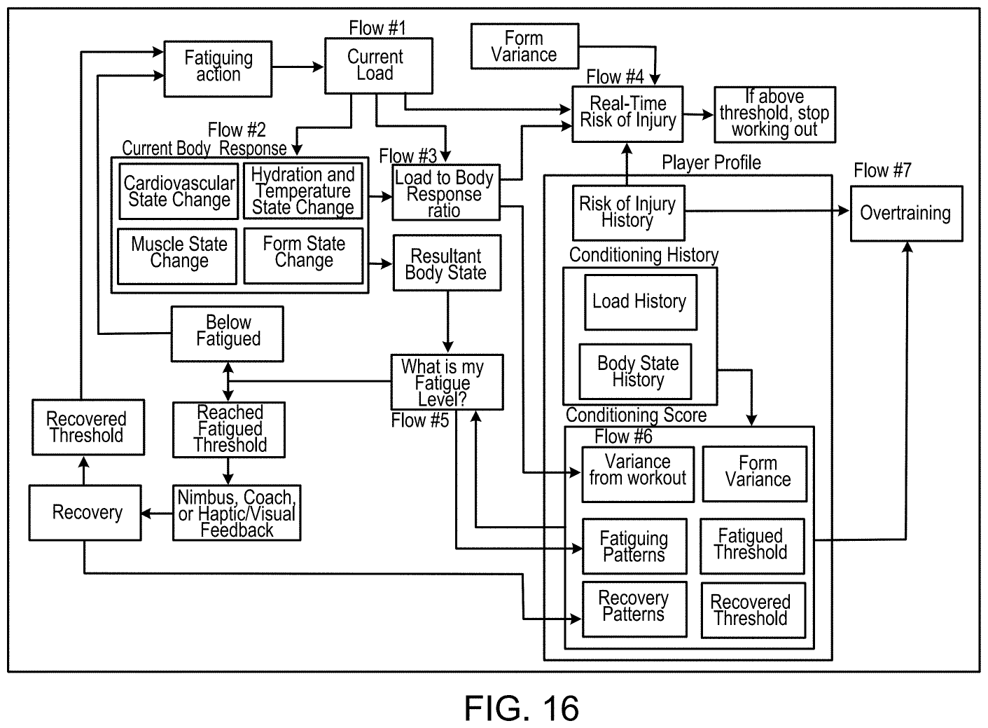

FIG. 16 shows a block diagram of a process to determine a body state of an athlete participant.

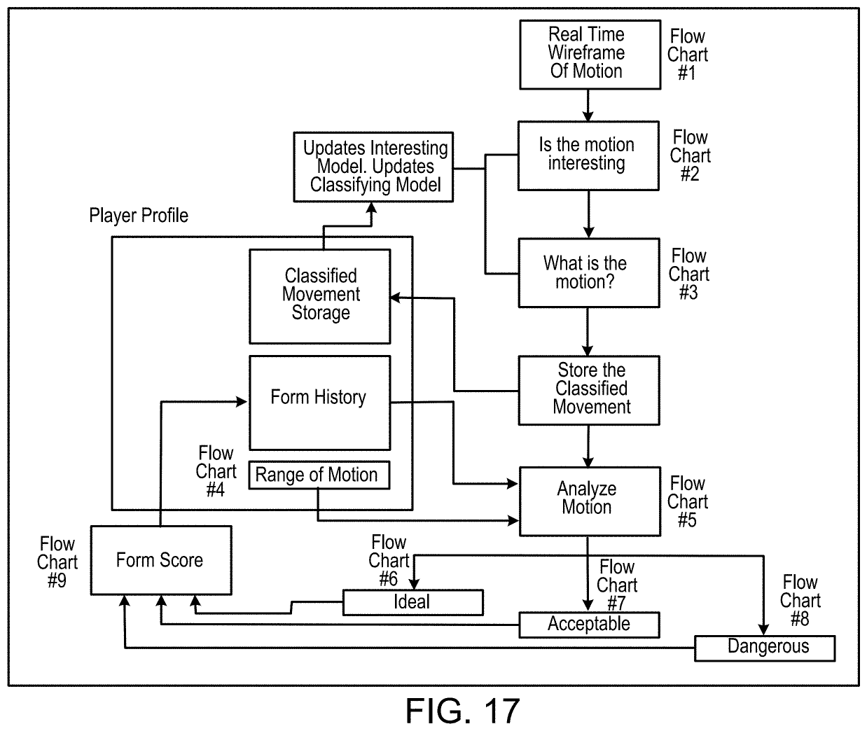

FIG. 17 shows a block diagram of a portion of analysis steps performed by the athlete technology.

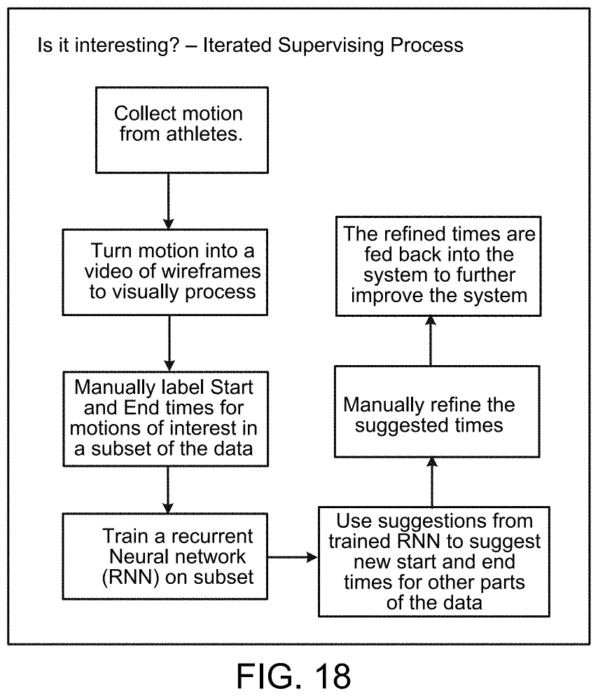

FIGS. 18 through 23 are block diagrams.

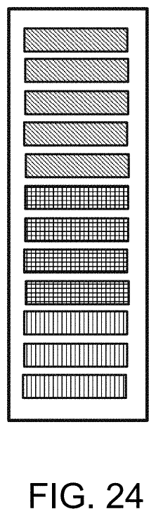

FIG. 24 is a user interface diagram.

FIG. 25 is a front view and back view of an athlete garment.

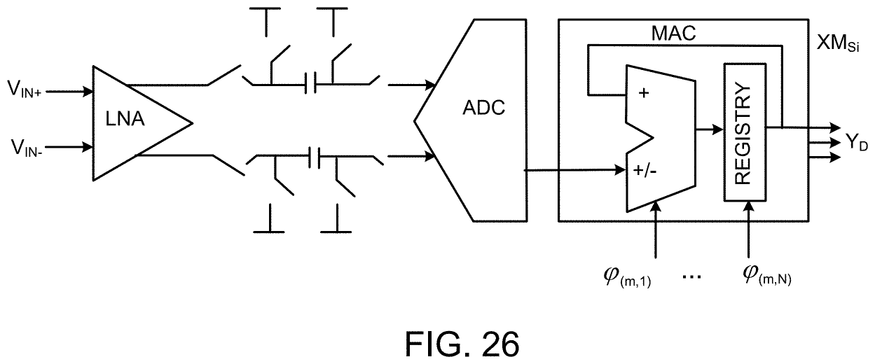

FIG. 26 is a block diagram.

Implementations of the athlete technology provide holistic processing of a wide variety of athlete data derived from sensors in the athlete garment worn by the athlete participant (or a group of them) and operated in a variety of modalities to acquire a variety of types of athlete data.

Because of the immediate, rich, and relevant quality of the feedback provided to the athlete participant and athlete stakeholders, the muscles and bodies of the athlete participants will start to be understood and perceived in a new way and the capabilities and limits on the athlete physical attributes and athlete performance factors will be more intuitively and completely understood, thus yielding improvements not currently possible.

In some implementations of the athlete technology, athlete stakeholders, while observing athlete participants engage in athlete activity, can provide real-time comments (e.g., human insights) on athlete physical attributes, athlete activities, and athlete performance factors for a particular athlete participant, or can highlight a certain activity or metric, alerting others to potential problems or investigable patterns. Among other things, this capability allows the participant stakeholders to understand each other's interests and knowledge and will make for a more cohesive group of athlete participants and athlete stakeholders.

### Sensors and Athlete Data

Among the sensors used and the athlete data acquired by the athlete technology are sensors and athlete data associated with the cardiorespiratory system and the musculoskeletal system of athlete participants.

Cardiorespiratory System and Metrics

Among the functions of the cardiorespiratory system are the drawing of air into the lungs, the binding of oxygen contained in the air to hemoglobin in red blood cells, and the pumping by the heart of oxygenated blood to the rest of the body. Important metrics of the functioning of the cardiorespiratory system include the respiration rate, that is, the frequency at which the athlete participant's lungs expand, the amount, that is, the tidal volume of air drawn in per breath, the heart rate, that is, the frequency of the heart's contractions, the heart rate variability, that is, the expected variation in heart rate, and the oxygen content in the body, that is, the percentage of muscle oxygen relative to blood oxygen.

Some implementations of the technology use a holistic combination of metrics and analysis of the overall cardiorespiratory system of an athlete participant to provide useful feedback to the athlete participant to enable the athlete participant to stabilize or improve athlete activity, athlete physical attributes, or athlete performance factors. Among other things, the metrics and analysis of the overall cardiorespiratory system can include a variety of factors indicative of the oxygen delivery dynamics of the cardiovascular system.

We use the word “holistic” broadly to include, for example, across two or more (or all) scales and degrees of connection. Data relationships can occur as direct, raw data relationships across permutations or combinations of sensor data, for example, when one calf's acceleration is in the net forward direction the other calf's acceleration is in the reverse direction. This relationship occurs at a low level of abstraction. Aggregate body motion (a highly constructed metric) as it relates to heart rate variability skewness is a relationship at a greater scale.

ECG

One measurement regime useful in understanding functioning of the cardiorespiratory system is electrocardiography (ECG). Like all muscle, cardiac muscle (the heart) produces an electric potential when tensing because a flood of calcium is released by the sarcoplasmic reticulum to trigger physical contraction of the muscle cells. However, the heart (hopefully) contracts in a coordinated and timely manner. To accomplish this, cardiac myocytes (muscle cells in the heart) also conduct a synchronized, nerve-like action potential. That action potential involves the motion of potassium, sodium, and chlorine ions. The multiple potential difference sources and the synchronization produce a strong and iconic signal representing heart activity. A prominent portion of the signal is the R-wave which corresponds to the contraction of the left ventricle.

Measurements of the heart beat are obtained using ECG, which measures the action potentials as the difference in voltage between electrodes positioned on the skin. Unlike skeletal muscles, the heart conducts nerve signals across the muscle and heart muscle cells contract in unison. In a typical ECG configuration, the electrodes used to measure these nerve signals are the inner, passive electrodes on the skin. The signature action potential of the heart is reliable and reliably shaped as illustrated in a classic ECG wave shown in FIG. 1 (see https://en.wikipedia.org/wiki/Electrocardiography).

In FIG. 1, the P wave represents the first wave of action potentials that travel along fibers of the heart top down to synchronize myocardial cells. The QRS complex is a spike that represents the myocardial cells contracting from top down and sending signals to their lower neighbors to begin contracting. The T wave represents the heart recharging bottom up to ready the next heartbeat.

Although abnormalities in the shape of the ECG wave are important clinically, some implementations of the athlete technology focus on the time interval between peaks of successive R waves (RR interval). The RR interval is an accurate measure of the time between heartbeats. In some examples, after collecting and preprocessing the ECG signal, the athlete technology discards all information except the RR interval. The reciprocal of the mean RR interval is the mean heart rate that is commonly reported by ECG devices.

HRV

Heart rate variability (HRV), a constructed measure of the variance of the RR intervals, is of interest to athlete stakeholders such as coaches and physiologists. One commonly accepted metric of the HRV is:

HRV(t)=StDev[RR(i)−RR(i−1)]/Mean[RR(t)] for i within the last 10 beats

The variance of successive beats is commonly used, and the normalization by the mean RR interval suggests the relative variability of the heart rate. The value of HRV is dependent on the intensity of athlete activity: the more intense the athlete activity the lower the HRV. As athlete activity stops and the athlete participant gathers breath, the HRV slowly rises to normal, resting values.

Although HRV is a metric of interest for understanding physiology of athlete activity, the athlete technology also takes account of the importance of statistical measures of these RR intervals that are more complicated than standard deviation. For example, skewness of successive RR intervals, the measure of the difference between the median RR interval and the mean RR interval, is a third-order statistical measure while mean and standard deviation are first and second order respectively. In some implementations, combining the metric of skewness with other physiologically relevant metrics enhances the effectiveness of the athlete technology in helping athlete participants to make improvements of one or more of the athlete physical attributes and athlete performance factors.

In examples of the athlete technology, RR interval data (e.g., one type of athlete data) can be acquired and stored whenever an athlete participant is engaged in athlete activity and is instrumented, for example, using athlete sensors (discussed below) in an athlete garment. By storing the RR interval data over time, and by processing it to derive statistical RR interval metrics, one or more athlete performance factors can be derived, analyzed, reported, and used in other ways, for example, to help the athlete participant to understand and make improvements in one or more athlete physical attributes or athlete performance factors. For example, an athlete participant's ability to restore normal HRV after athlete activity can be derived at a given time and compared to that athlete performance factor for one or more of the peers of the athlete participant or compared to that athlete performance factor for the same athlete participant at prior times. In some instances, the athlete technology can establish a threshold of HRV restoration rates after exercise below which a player can be presumed to be too fatigued. In some cases, if a player's HRV takes more than five minutes to rise to their resting value, the athlete technology can determine they are likely too spent for further training to be safe.

Respiration Metrics

To measure the respiration rate of the athlete participant, in some examples, the athlete technology measures the impedance (resistance to electric current) of the thorax. The measurement is sensitive to changes in electrical conductivity. By injecting known amounts of current and measuring the resulting electrical potential field at points on the boundary of the body, it is possible to invert such data to determine the conductivity or resistivity of the region of the body probed by the currents. This method can also be used in principle to image changes in dielectric constant at higher frequencies, and therefore is sometimes called impedance tomography rather than conductivity or resistivity tomography.

The athlete technology applies this method based on placement of four electric contacts on the body, two on each rib cage, to form an inner pair and an outer pair. The outer pair drives a current across the thorax at a specific frequency that the human body is unlikely to generate, for example, 100 kHz, and the inner pair measures the voltage across the thorax at that 100 kHz frequency. This type of measurement is called active measurement, because it involves injecting energy into the measured system. To reduce noise, one pair of electrodes is driven actively, sending current into the tissue, and the induced voltage is measured passively using the other pair versus sending current and measuring voltage on the same two electrodes.

The complex ratio of the measured voltage divided by the driving current is the impedance of the torso, and the impedance changes rhythmically with the respiration cycle. As the lungs expand the thorax expands slightly and tissues are stretched apart. The increased distance between left and right rib cages along with the thinning of the connective tissue between, increase the impedance between rib cages. Because of how closely the impedance changes track the respiration cycle and vice versa, analysis of the thoracic impedance can indicate the respiration rate, how sharply the athlete participant inhales or exhales, how long air remains in the lungs, and other respiration information. This athlete data can be useful in a variety of ways.

In addition to being indicative of the respiration cycle, the impedance signal also carries a higher frequency component that depends on cardiac output (heart rate time stroke volume during a period of time). That is, the calculated impedance signal carries a high frequency change, between 50-180 hertz. Heart beats do not affect thoracic impedance as much as does breathing, but because the heart rate is faster than the respiration rate, the athlete technology can isolate the effect of heart rate and deduce the stroke volume, that is, the volume of fluid the left ventricle expels per contraction. Stroke volume is a strong predictor of athlete performance factors. A frequent (e.g., daily) calculation of stroke volume indicates improvement of fitness and other athlete performance factors providing, for example, a way for athlete participants to visualize improvements in their fitness resulting from training.

Cardiac Output, Stroke Volume

Cardiac output is the volume of blood pumped by the heart during a time interval. Typically, the time interval is a minute. For example, the stroke volume at rest in the standing position averages between 60 and 80 ml of blood in most adults. The measurement of cardiac output is useful both for establishing an athlete participant's initial cardiovascular state and for monitoring the response to various exercises and to an athlete participant's return to exercise after a prolonged period of reduced exercise or lack of exercise. Patterns can be used to adjust the athlete model to reduce prescripted exercise loads and update risk of injury calculations.

Stroke volume is a more complex metric to measure compared to heart rate.

To measure heart stroke volume, electrode pairs are placed level with the xiphoid process, on the sides of the thorax, posteriorly offset in the sixth intercostal space, where the arm does not brush against the electrodes. A 100 kHz alternating current is applied across the two outer electrodes and the voltage drop is measured across the inner electrodes. The ratio of the applied current to the measure voltage drop is used to calculate the impedance. 100 kHz is chosen to maximize the dynamic range of measured signals, increasing the resolution of the measurement system. International standards are set to regulate the total current one can safely inject into the human body, and the power of the driving circuit is maintained well below the 10 mA limit established in ISO 60101-3.

In some implementations, there may be extra driving and sensing electrode pairs to improve the signal.

In some implementations, the athlete technology may apply bioimpedance spectroscopy which involves injecting a sweep of frequencies into the region of interest. In some cases in which it is applied to the thorax, it will aid in frequency localization which is important for separating cardiac and respiratory signals. In some cases in which it is applied to an electrode pair with one electrode located on the forearm and the other electrode on the calf, bioimpedance spectroscopy allows for accurate relative and absolute hydration measurements. The connection between upper and lower garments creates a common ground which enables the whole body hydration measurement.

FIG. 2 (see https://www.ncbi.nlm.nih.gov/pmc/articles/PMC4209026/) shows known circuits for measuring respiratory impedance and cardiac impedance.

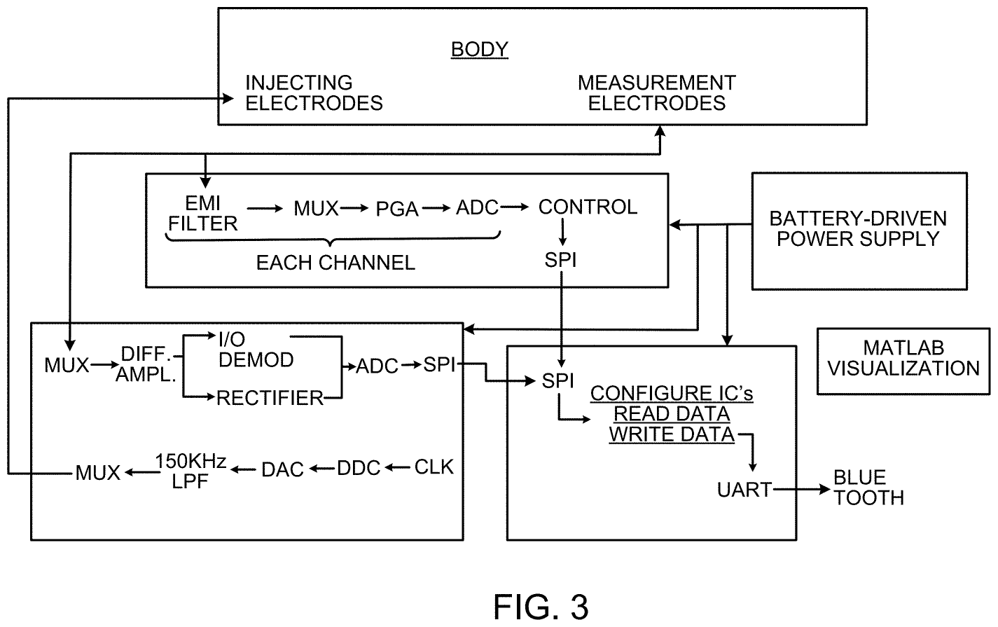

FIG. 3 shows (seems https://docksci.com/development-of-a-wearable-multi-frequency-impedance-cardiography-device_5a71f08ed64ab27399b2bf23.html) known circuits for measuring ECG and ICG using available AFE4300 and ADS1298 integrated circuits.

Musculoskeletal System

Near Infrared Spectroscopy

One of the detailed feedback loops at work in the body of the athlete participant and useful in the athlete technology relates to the delivery, storage, and use of oxygen in muscle tissue. The efficiency of this loop limits what the muscle can achieve over time.

In implementations of the athlete technology, near infrared spectroscopy can be used to measure the body's concentrations of metabolically significant compounds such as the heme-containing proteins hemoglobin and myoglobin. Hemoglobin shuttles oxygen from the lungs to the rest of the body, and myoglobin stores oxygen in muscles. Hemoglobin and myoglobin have different reflective responses to light that change when binding oxygen, specifically the frequencies of light they reflect most shifts. By shining frequencies of light having the biggest discrepancies between oxy-hemoglobin (oxygenated hemoglobin) and deoxy-hemoglobin (unoxygenated hemoglobin), for example, the athlete technology can calculate the ratio of oxygen binding to non-oxygen binding hemoglobin.

Although the calculation of the ratio can be done reliably at many light frequencies, good frequencies fall in the near infrared range. Human bodies are largely transparent to such frequencies, excepting hemoglobin and myoglobin. For techniques that use visible green light, the absorption of the test light depends on the skin color of the patient. Near-infrared light avoids this difficulty and also use of high enough frequency not to be misled by the black-body radiation of the body. The average wavelength emitted by a person at 310 kelvin (healthy body temperature) is 10000 nanometers, while the wavelengths we use in some implementations of the athlete technology are an order of magnitude less, ˜700-1000 nm.

The ratio of blood oxygenation levels of oxy-hemoglobin and deoxy-hemoglobin is fairly static in the blood stream of healthy people, but at the muscle, oxygen dissociates from the hemoglobin molecule and binds to myoglobin. So locally at the point of measurement during athlete activity, the ratio rapidly changes. The ratio is also dynamic because myoglobin is a storer of oxygen and not a mover of oxygen. When an athlete participant engages in athlete activity anaerobically their oxy-myoglobin turns to deoxy-myoglobin depleting the oxygen stored in the muscles. When the athlete participant reduces the intensity of athlete activity such that the oxygen demands of their muscles are lower than the rate of blood oxygen delivery, the oxy-myoglobin concentration spikes up to renormalize. In practice, it is not easy to isolate the measurement of myoglobin. However, myoglobin comprises roughly 10% of the hemoglobin signal. In some implementations of the athlete technology, near-infrared spectroscopy is applied at the site of muscle and tuned to frequencies most reflected by oxy- and deoxy-versions of myoglobin and hemoglobin and the resulting signal is treated as predictive of fatigue in the body of the athlete participant. Information about calculating SmO2 can be found at https://www.osapublishing.org/DirectPDFAccess/8222D300-A88B-9066-8D49F480FA48530E_142888/oe-15-21-13715.pdf?da=1&id=142888&seq=0&mobile=no, incorporated here by reference.

In some cases, this technique includes shining specific wavelengths of light through the skin into the tissue and measuring the fraction of light that returns (as reflected) to a fiber bundle. Every molecule has a distinct absorption or reflectance curve: the fraction of light absorbed or reflected at each wavelength. Within the curve, there are peaks and troughs at wavelengths at which it is useful to make measurements.

In some examples, the athlete technology includes light emitting diodes (LED's) held on inside surfaces of garments and positioned to face muscle tissue of the athlete to send near infrared light of selected frequencies into the rectus femoralis muscle and then collect the reflected light through a fiber bundle to a photodiode that produces a current when it receives the reflected photons. The sensor has the fiber optic bundle at the center, encircled or flanked on both sides by LED's that emit light at the wavelengths associated with tissue water content, oxy-hemoglobin, and deo-xyhemoglobin. The purpose of opposing light sources is to reduce error in the path-length measurement and to ensure even illumination of the tissue. As the photons from the light source penetrate the tissue, the photons perform a Monte Carlo random walk as the light interacts elastically through the tissue. The path length is the statistical average of these random walks through the material as a function of its material properties. The athlete technology in some cases uses optical fibers at the center of the sensor to reduce the acceptance of light directly lateral to the end of the fibers on the surface of the skin. We choose the rectus femoralis, a major muscle group in the thigh, for its accessibility, its size, its vascularization, and for its physiologic relevance as most activities our athlete participant's engage in straining it in some way.

In some examples, the athlete technology does reflectance sampling using light at 760 nm and 850 nm. In some cases, the light could be at other wavelengths that are determined to provide better results. In some instances, the light sent and received can be treated as vectors of light intensity values at different frequencies. In addition, the concentrations of relevant chemicals in the body that absorb and re-emit those frequencies (for example, oxy-hemoglobin, -deoxyhemoglobin, oxy-myoglobin, deoxy-myoglobin, and -cytochrome, and deoxy-cytochrome) as vectors and those chemicals' individual reflectances at certain frequencies could be considered as a matrix according to the following equation:

R\hat{ }(−1)r/s=k*c

where r/s is the ratio of received light to sent light at each frequency, k is a parameter that describes path length and attenuation, R is a matrix of chemical reflectances, R\hat{ }(−1) is the matrix inverse of R, and c is the vector of chemical concentrations. The equation cannot be solved for the absolute concentrations of these chemicals without estimating the parameter k. However ratios of linear combinations of chemical concentrations can be determined without any knowledge of k:

kc1/kc2=c1/c2

Therefore it is possible to determine the ratio of oxy-myoglobin to deoxy-myoglobin and oxy-hemoglobin to deoxy-hemoglobin. These ratios are reliable measurements of the amount of oxygen muscle tissue has stored. Determinations also can be made of the cytochrome molecule most commonly used in the mitochondria of cells. For example, the ratio of total cytochrome to total myoglobin ought to represent the mitochondrial density of muscle tissue unless myoglobin is rapidly produced in the cells.

The profile over time of oxygenation of muscle tissue reflects the fitness of the muscle, for example, the vasculature the athlete participant has built by training to support the muscle's need for oxygen and the general condition of the athlete participant's cardiovascular system. In general, a muscle's fitness is defined be its strength, endurance, and flexibility. Muscular strength is the maximum force that can be generated in one instance. Muscular endurance is the ability of a muscle (or group) to execute repeated contractions (perform work) over a period of time causing muscular fatigue. Deriving a profile of the athlete participant's muscle efficiency in this sense contributes to a general model of fatigue and can also yield a potent analysis of individual and population variation of this attribute. For example, an athlete participant who begins training after recovering from an injury in one leg expects to improve the use of one leg more than another. Plotting the previously injured leg's ability to replenish stored oxygen as compared to the opposite healthy leg provides a compelling graphic demonstration for the athlete participant and other athlete stakeholders.

In overtraining, accumulated tears and oxidative stress on muscle tissue may render it unable to contract. The analysis described above could detect this effect by identifying anomalies in relative chemical concentration. For example, in some implementations of the athlete technology, charting the equilibration of relative concentrations of chemicals in muscle tissue may help athlete participants and other athlete stakeholders to manage productive and effective recovery.

Muscle Contraction

Another detailed feedback loop (control system) at work in an athlete participant's body and useful in the athlete technology relates to the contraction of muscles in response to signals from the brain, the effect of the contractions on other elements of the musculoskeletal system, and the motions produced by the contractions.

We use the term “musculoskeletal system” broadly to include, for example, the bones and bone geometry, ligaments, tendons, and muscles.

Muscles typically are connected to bones through tendons. The tendons can absorb energy by stretching to prevent quick muscle contractions from creating stresses at the connection points of the muscles to the bones. The tendons capture and dissipate some of the impulse created by the contracting muscle, returning the energy of the impulse to the muscle later in the movement cycle.

The shapes of the bones are taken to be static; the dynamic parts of the musculoskeletal system are taken to be the tendons, ligaments, and muscles, and combinations of them. Because the brain cannot consciously control the behavior of tendons and ligaments, they are modeled as passive spring-like structures, passively storing and releasing energy during operation of the musculoskeletal system.

To initiate a contraction of a muscle, the brain sends a signal through nerves and delivers it to the muscles. The neurons that interface with the muscle are called motor neurons. These motor neurons directly innervate a number of muscle cells directly. The motor neuron and the innervated muscles cells are called a muscle unit. The motor neuron delivers the contracting signal to the muscle cells as a motor unit action potential (MUAP). Clusters of muscle units in a muscle are called innervation zones (IZs) and may be numerous. In some implementations of the athlete technology, information about activity at the IZ having the greatest contribution in beginning a contraction of a particular muscle is used for various purposes. Often that IZ is the largest cluster.

During a muscle contraction, the fibers within the muscle pull against plates in the fiber, bringing the plates closer together and shortening the fiber's length. As the fiber shortens, conservation of the volume of the fiber requires the fiber to thicken, producing the familiar curved muscle bulge. The peak of this resultant curve is called the muscle head.

Because features of muscle contraction are important to understanding athlete performance factors, in some implementations the athlete technology uses various measurement modalities for sensing and analyzing information about muscle fitness, muscle contraction, and musculoskeletal motion and uses the information for a wide variety of purposes and applications.

IMUS

One of the measurement modalities used in some implementations of the athlete technology relies on inertial measurement units (IMUS) held within or on athlete garments to track acceleration, force, and angular rotation of parts and segments of the musculoskeletal system. In some cases, 10 IMUs are used although other numbers of IMUs can also be effective. An IMU is a micro-electro-mechanical (MEMS) device that contains an accelerometer, a gyroscope, and a magnetometer for measuring specific force, angular rate, and magnetic field, respectively. These three sensors are often used together because the data from each sensor can be used to correct measurement drift and errors in the other sensors. The sensor data is combined using a commonly available data fusion algorithm executed on a co-motion processor. In some implementations of the athlete technology, each of the IMUs placed on the body includes such a co-motion processor to increase computational efficiency. In some implementations of the athlete technology, the IMUs may not include or may not use embedded co-motion processors but instead may send sensor data to another device for processing without using a co-motion processor to process it locally or remotely.

The result of the data fusion effected by the co-motion processor is athlete data in the form of a drift-corrected orientation vector called a quaternion (four numbers) and raw accelerometer, gyroscope, and magnetometer data (three numbers each). In implementations using 9 IMUs, the output data from the IMU sensors therefore includes an output data set of 9×13=117 output data elements for each execution cycle of the co-motion processor. Each of the output data sets can be sent immediately to another device or may be stored temporarily while awaiting a later transfer of one or more of the stored output data sets to another device. The execution cycles of the IMUs could occur at a variety of rates, for example, in a range of about 150 Hz to 250 Hz.

Each IMU can also contain a temperature sensor the output of which can be used to internally calibrate the sensor as the materials in the IMU that constitute the sensors are sensitive to temperature changes. In some implementations of the athlete technology, the temperature sensor is also used to measure body temperature at the sensor placement site. Body temperature at one or more sensor placement sites can serve as a vital sign for deriving (for example by holistic analysis) one or more athlete performance factors such as the athlete participant's fatigue or likelihood of injury. Among other things, the athlete technology can use body temperature data to identify and alert athlete participants or athlete stakeholders of impending lightheadedness and eventual heatstroke when body temperature at one or more sensor placement sites rises above a threshold, for example, 105 degrees Fahrenheit peripheral body temperature. Body temperature is a vital sign useful in a holistic analysis of athlete physical attributes or athlete performance factors such as the athlete's fatigue or likelihood of injury. Simple thresholds can be selected and used as the basis of alerting athlete participants of impending lightheadedness and eventual heatstroke. A threshold of 105 degrees Fahrenheit for peripheral body temperature is reasonable.

Kinematic Wireframe

Another musculoskeletal system measurement modality relates to a use of kinetic wireframes. In some implementations of the athlete technology, a 3D model of the athlete participant is produced by photogrammetry (as described below). Based on the 3D model, a determination is made of favorable sensor placement sites for IMUs on athlete body segments (see FIG. 5, reference http://old.cescg.org/CESCG-2000/RFilkorn/index.html). By appropriate selection of the athlete body segments, the 3D model can produce accurate animations of the athlete participant using output data sets from the IMUs. An avatar represented by this animated 3D model can be used in a biomechanical model (BMM) of the athlete participant. In some implementations, the wireframe can constitute a set of straight lines each of which connects a pair of locations at which the IMUs have been placed. The sensor placement sites for the IMUs are in fixed locations relative to the body (except to the extent that the garment on which the IMUs are held may shift relative to the body).

We use the term “athlete body segment” broadly to include, for example, a portion of a body of an athlete participant extending between two points on the body. The portion of an upper arm extending between one point at the shoulder joint and one point the elbow joint could be considered an athlete body segment, for example. In some cases, an athlete body segment is a relatively rigid portion of the body and the points at which respective athlete body segments meet our joints or relatively flexible portions of the body.

In a sense, the sensor placement sites of the IMUs form an IMU wireframe corresponding to the skeleton and corresponding to the computer-generated 3D model wireframe that is derived from the IMUs. A computer-generated wireframe is a basic form for representing kinematics and therefore is sometimes called a kinematic wireframe.

In some implementations of the athlete technology, the kinematic wireframe is used to analyze patterns of motions of the athlete body and athlete body segments of the athlete participant to identify, quantify, and provide athlete data and athlete insights on athlete physical attributes and athlete performance factors such as fatigue in a changing gait, or injury biomechanics when an athlete participant is injured. In the latter case, the athlete insight is useful for rehabilitating the injured part of the body. Most human motion is a complex combination of linear and angular motion components. Because linear and angular motion can be considered pure forms of motion, it is sometimes useful to break complex motions into their linear and angular components when performing an analysis. Additional information about this approach can be found at https://accessphysiotherapy.mhmedical.com/content.aspx?bookid=1586&sectionid=99981270, incorporated here by reference

Machine Learning and Athlete Motions

In some implementations, the athlete technology uses machine learning to process the IMU data sets to classify athlete motions represented by the IMU data sets as they happen or after they have happened or both. In some cases, the athlete technology uses a recurrent neural network (RNN) and an iterated supervising process to train a model to detect if a period of body motion of a wireframe is of interest.

We use the term “athlete motions” broadly to include, for example, any action in which one or more parts of an athlete's body changes position, orientation, or location, for example. Athlete motions can be simple or complex. Athlete motions can involve one or more than one athlete body segment. Athlete motions can occur at once or in sequence.

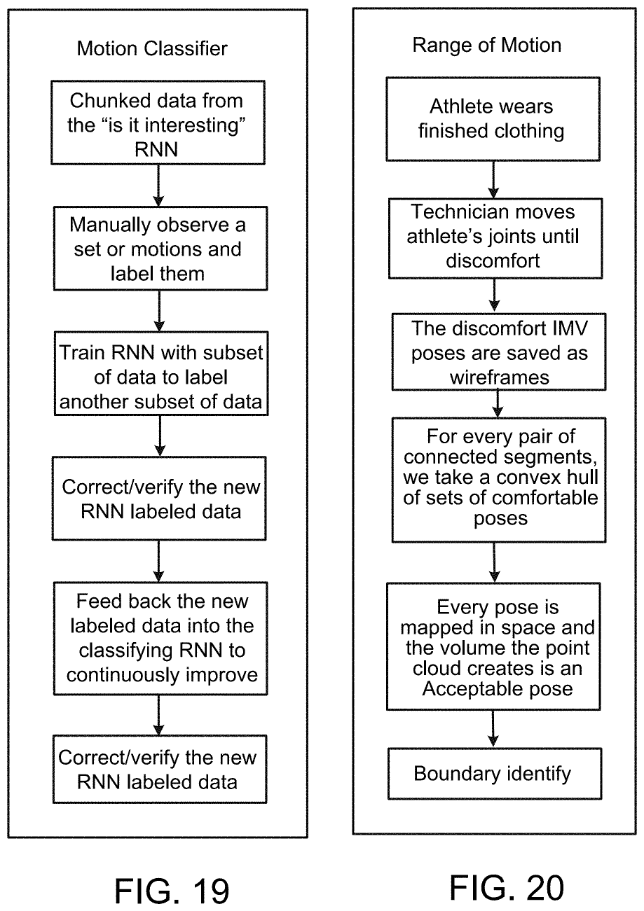

As shown in FIGS. 18 and 19, to train the RNN, the athlete technology collects a large group of examples of data sets corresponding to motions of wireframes of athlete participants engaging in a variety of athlete motions of interest. Manually, the beginning and ending moments of each of the athlete motions of interest are marked for a small portion of the large group of examples. Among the motions that are labeled are traditional resistance training and weight-lifting exercises, and (in the case of basketball) lay-ups, free throw shots, 3 pointers, 2 pointers, jab steps, jump shots, fake shots, and if an athlete participant's body was interfered with during a shot. After manually marking the beginning and ending moments of the motions for the small portion of the large group of examples, the RNN is trained to predict whether each motion represents an activity of interest. For another small portion of the large group of examples, the RNN's predictions are used to speed up a manual marking process. By tweaking the RNN's predictions slightly by hand the athlete technology can quickly and accurately construct the next small portion of the large group of examples for refining the RNN. This process continues until an RNN can reliably detect the beginning moment of an interesting athlete motion. After collecting and labeling athlete motions of a small number of athlete participants, additional manual labeling is not necessary, except to label a new type of athlete motion that has not been previously labeled. Labeling an athlete motion has the effect of making that athlete motion interesting (of interest) to the athlete technology.

In some implementations of the athlete technology, another RNN network is used to identify a current athlete motion of interest. This second RNN analyzes the current athlete motion and classifies it (for example, in the case of basketball, as a free throw, landing, or pass). This RNN can be trained using a similar technique to the training of the first RNN, that is, first using unassisted manual labeling and then assisted manual labeling. In other words this second RNN is a neural network which is used to tell which athlete motion the athlete motion of interest was. The trainers or other athlete stakeholders and the athletes can initially propose new athlete activities and apply labels after performing the new athlete activities. Results can be added to an athlete motion database. These “homebrew” athlete activities and corresponding labels can be offered to other athlete participants and athlete stakeholders. Among other things, this allows them to adjust start and end times to train the athlete technology.

The first (relatively light-weight) RNN will run continuously and turn on the second (heavier) RNN to classify the current athlete motion after it has happened. Based on the output of the first and second RNNs, a profile of an athlete participant's motions can be generated to identify, quantify, or analyze athlete performance factors, such as the variance of form of a given athlete participant or bad form of an athlete participant relative to a standard or relative to other athlete participants.

Poses and Trajectories

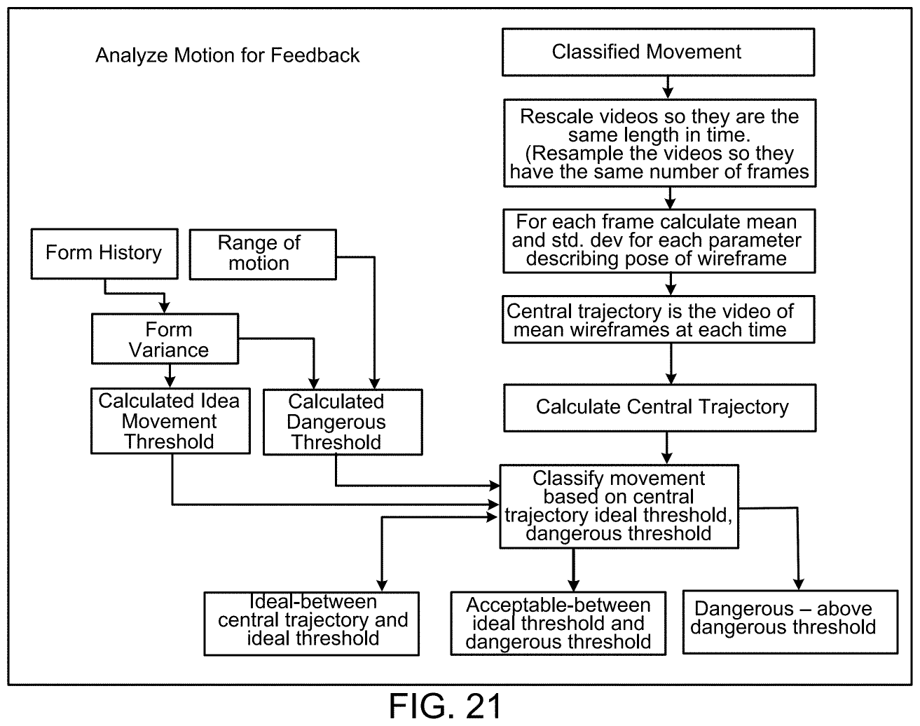

As shown in FIG. 21, a given frame in a sequence of frames constituting a moving kinematic wireframe is sometimes called a pose. A sequence of poses, for example, within an athlete motion represented by successive frames of a kinematic wireframe, is sometimes called a trajectory. Trajectories can be grouped by the similarity of athlete motions and a mean (or other metric of centrality) trajectory can be constructed for the group of trajectories. This mean trajectory represents the most likely trajectory for the given athlete motion for a particular athlete participant or a group of athlete participants. Ideally, the mean trajectory for a given athlete participant corresponds to the typical form trajectory for a particular athlete motion. The athlete technology, in attempting to help to change an athlete participant's biomechanics to more optimal trajectories, would try to move away from this mean trajectory. What makes the mean trajectory special is that this is an important trajectory for creating a proper workout plan and seeing in what areas that trajectory's component forces can be altered or improved. Large and immediate deviations from the mean trajectory are usually bad because the proprioception of that changed movement hasn't yet been refined or committed to memory, potentially leading to an injury. Using a handpicked function on the standard deviation of the poses with respect to each body segment on the wireframe, or perhaps a handpicked volume, the athlete technology can identify a volume (e.g., a bubble) around the mean trajectory of that body segment that represents all possible trajectories for that body segment that are typical.

In some implementations, the athlete technology can determine a rate at which an athlete participant's trajectory deviates from their mean trajectory or other central trajectory. Such a rate can be used as an input or a weighted input for computation of an athlete performance factor such as a measure of fatigue. It is also possible to predict if the athlete participant will deviate from their central trajectory by using other sensor data. Among other things, the athlete technology can also identify which body segment of the wireframe, for example, which limb or joint, was the first to deviate from the central trajectory. This information can be stored, analyzed, or communicated to the athlete participant in an appropriate message or by another form of alert. For example, the message could say: “Your elbow flared outward and changed your form.” Given physiological constraints on the athlete participant's skeletal wireframe, such as the dorsal angle between the upper leg and the lower leg being less than 180 degrees, the athlete technology can set thresholds for the wireframe past or near which damage to the athlete participant's body would be expected. The thresholds relate to when a muscle head may be overextended (strained).

In other words, the athlete technology can use the rate at which athlete participant deviates from his central form as a weighted input to a measure of fatigue. The athlete technology can try to predict if the athlete will deviate from their form by using the rest of the sensed athlete data.

The athlete technology can also compare the trajectories of an individual athlete participant to the trajectories of a population of athlete participants. Athlete insights can be developed from any determined differences. For example, some differences are unconventional, others are indicative of excellent quality, and some are bad or risky. Athlete insights can be developed based on information provided through a user interface by the athlete participant or athlete stakeholders (such as trainers).

sEMG and Laplacian Electrodes

Another musculoskeletal system measurement modality used by the athlete technology involves measuring electrical signals (sEMG) from muscles. Muscles begin contraction after receiving an electrochemical signal from innervating neurons. The contraction process in the muscle involves a (relatively) massive flow of calcium ions manifesting on the skin as small potential differences.

Measuring those potential differences in the signal at the skin provides information about the muscle contractions near the skin. Although the signal measured mostly originates in the muscle fibers directly underneath the point of measurement on the skin, the signal is muddied by signal components from fibers farther away. The fibers directly underneath the point of measurement and the fibers farther away may not produce signals that are in phase. The fibers at different distances from the point of measurement on the skin may produce identical signal components, but the signal components from different fibers may appear at different times. The athlete technology uses Laplacian electrodes to compensate for the timing differences.

A conventional method for measuring the electrical activity of a muscle of interest is by measuring the voltage difference between two conducting pads placed on the skin in the vicinity of the muscle. In a concentric, or Laplacian, electrode one of the conducting pads is a ring (or two or three rings) of conducting material surrounding a central conducting pad. The signal constituting the voltage difference between the central pad and one or more of the ring pads is recorded. The voltage difference recorded in the conventional method is a unidirectional flow of electricity along the surface of the skin from one pad to the other. In the concentric Laplacian electrode electricity flows up to the skin from the muscle tissue directly below the central pad of the Laplacian electrode and then radially along the skin from the central pad to the ring pad in a fountain-like configuration.

In measuring the fountain-like flow of electricity from muscle to skin, the ring pad is most sensitive to the tissue below the Laplacian electrode, which reduces crosstalk in which muscle tissue far from the location of the Laplacian electrode contributes to or muddies the signal. The use of Laplacian electrodes enhances the confidence that the signal detected by the sensor comes from the tissue at the sensor placement site. The Laplacian electrode is essentially a spatial filter. By increasing the number of rings and by optimizing those rings' widths (inner radius to outer radius), localization of the measurement can be tuned to the point of measuring a signal motor unit of the muscle. The Laplacian electrode's dimensions focus measurement at a dipole at a certain depth below the electrode, which can also be tuned. Placing the Laplacian electrodes directly above identified innervation zones of the muscle group of interest enhances the likelihood that the signal gathered accurately reflects electric activity of the muscle group of interest. As the name suggests the concentric model can be made more sophisticated by adding additional ring pads around the central pad to better approximate the Laplacian of the electric field at that central pad.

The Laplacian electrode is placed over the innervation zone near the nervous system's sent signal to improve the dynamic range and amplitude of the measurement. Because sEMG (surface electromyography) is almost always performed using a bipolar electrode, fatigue is typically estimated by interpreting a signal morphology specific to a bipolar configuration. Analyzing the morphology of signals collected by Laplacian electrodes at sensor placement sites at innervation zones can provide more effective, accurate, and useful results.

In some implementations of the athlete technology, Laplacian electrodes can be implemented for sEMG sensing by weaving conductive thread into fabric of the garment to be worn by an athlete participant or by printing conducting ink to form the concentric pads, then connecting insulated wires to the pads to ferry the signals to analog circuits also attached to the garment. In some examples, the Laplacian electrodes can be created separately and then stitched into the garment.

In some implementations of the athlete technology, the material for the pads of the electrode can be the conductive polymer PEDOT:PSS (poly (3,4-ethylenedioxythiopene) polystyrene sulfate). In some implementations of the athlete technology, the material for the pads of the electrode can be a conductive polymer made with a ratio of carbon nanoparticles and PDMS (polydimethylsiloxane). Such polymers are cheap, highly tunable, ductile, and biocompatible. The specific fabrication method for the electrodes can depend on the implementation used for the sEMG sensors: weaving, stitching, printing, molding, or stenciling. In some implementations, combinations of weaving, printing and separate stitching could be used.

In some fabrication implementations, a stencil or a mold can define boundaries of the pads of the electrodes atop the surface of the fabric. A gel of the conductive material can then be laid down over the stencil or within the mold. The gel adheres to the fibers in the textile creating a washable athlete garment or portion of the athlete garment.

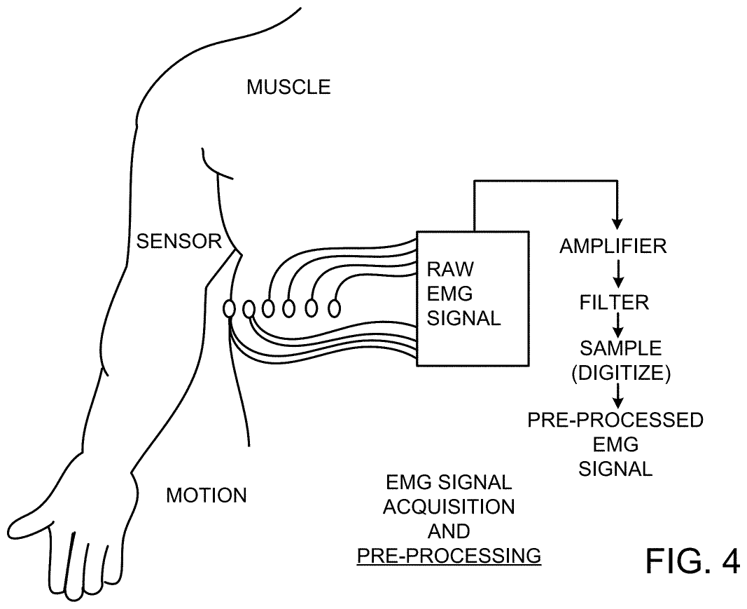

Therefore, Laplacian electrodes serving as sEMG sensors can be placed at innervation zones of muscle of the body to be used to detect, quantify, analyze, and report on athlete motions and athlete performance factors. Wires can connect the sensors to EMG signal acquisition and preprocessing circuitry. As shown in FIG. 4 (see https://www.researchgate.net/publication/262011093_A_Review_of_Control_Methods_for_Electric_Power_Wheelchairs_Based_on_Electromyography_EMG_Signals_with_Special_Emphasis_on_Pattern_Recognition,) the raw sEMG signals are received and then provided to an amplifying component, a filtering component, and then a sampling (digitizing) component to provide a pre-coded sEMG signal. The sEMG signal acquisition and preprocessing circuitry can be included in the athlete garment to be worn by the athlete participant. In some implementations, the preprocessed EMG signal can be forwarded to processing devices located off the athlete garment, such as in a mobile device, a workstation, a server, or a cloud platform, or combinations of them.

sEMG Electrode Placement Optimization

Electrode placement is important for obtaining accurate estimates of sEMG signals. A good location for sEMG electrode placement is between a muscle innervation zone (IZ) and a muscle tendon. However, the locations of the muscle IZs vary from athlete participant to athlete participant, so it is important to find the locations of these zones for every athlete participant. A traditional manual process of locating muscle IZs by visual analysis is time-consuming, inaccurate, and often not reproducible. Implementations of the athlete technology locate these IZs automatically and accurately. After an IZ is located, the athlete technology uses signal analysis to find the minimal crosstalk area (MCA). The MCA is the location where the noise coming from sEMG signals of co-active adjacent or inactive muscle is at a minimum. The athlete technology automatically and accurately identifies optimal sensor placement sites by finding both IZs and the MCA for each IZ. Sensor placement sites are optimized for various muscles in the body with varying resolution of signal. Some muscles include both major upper-body muscle groups including: pectorals, biceps, triceps, deltoids, latissimus dorsi (lats) and trapezius (traps), and major lower-body muscle groups: inner quadriceps, outer quadriceps, hamstrings, and gluteus (glutes).

Locating Muscle Innervation Zones for Sensor Placement:

A known process for locating muscle innervation zones for optimal sensor placement sites is set forth in https://journals.plos.org/plosone/article?id=10.1371/journal.pone.0167954 (incorporated by reference here) and includes four activities: image-preprocessing, image-segmentation using kernel graph cuts and watershed, pruning and region identification, and IZ detection and feature extraction.

sEMG Measurements Used for Muscle Fatigue Detection

Muscle fatigue detection is a useful application of sEMG in athlete participants. In the athlete technology, muscle fatigue is detected from the median frequency of the sEMG power spectrum. The regression slope of the linear regression of median frequency is an important muscle fatigue index. A more negative slope value represents a higher muscle fatigue condition.

Muscle fatigue is a complicated athlete performance factor that results from insufficient blood oxygen and nutrition. There are three types of fatigue: central fatigue, fatigue of the neuromuscular junction, and muscle fatigue. Muscle fatigue is of interest in sports medicine, as it gives an estimation of global fatigue of the athlete. There are three types of controlled muscle contractions: isotonic (maintain same force), isometric (maintain same position), and isokinetic (maintain same velocity). Isometric contraction is a static movement. The body segment of the athlete participant is held in a static position; isotonic contraction is a dynamic movement which is any regular complex movement that an athlete participant does in their day-to-day life. Local muscle fatigue can be continuously monitored by sEMG, using maximum isometric and isotonic contraction parameters. It can also demonstrate the biochemical and physiological changes in muscles during fatiguing contractions. The advantages of sEMG are non-invasiveness and real-time fatigue monitoring during athlete activity; it can also monitor the fatigue of a particular muscle that is highly correlated with biochemical and physiological changes in muscles during fatiguing actions.

The recorded sEMG is divided into many segments and a Fast Fourier Transform is performed.

The MF (muscle fatigue) of each segment is extracted. MF is defined as the frequency at which the accumulated spectrum energy is half of the total spectrum energy and p(f) is the power spectrum density of sEMG.

The window size of the sEMG segment is 30 seconds, and step size is 15 seconds. In order to quantify the distribution of MF during the examination of three stages, a linear regression analysis is applied to evaluate the muscle fatigue condition. The linear function is well known:

y=Ax+b

where y is MF and x is the time interval, A is the regression slope and b is the bias. The greater the muscle fatigue, the smaller the slope. We also used the correlation coefficient (r) to represent the stability of sEMG in terms of muscle fatigue, which is defined as:

$$r = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2} \sqrt{{\sum }_{i = 1}^{n} {( {y}_{i} - \overline{y} )}^{2}}}}$$

In this process, x is time and y is MF frequency. The more stable sEMG, the larger the r value. As muscle exercise time increases, MF increases. Parameters r and A describe the variability and degree of muscle fatigue.

Force Myography (FMG)

Another sensing modality used by the athlete technology is force myography (FMG). In FMG, a pressure sensor is placed on a muscle at a sensor placement site to measure force produced at the site. FMG is used in prosthetic control for detecting where an amputated limb is pressing within a prosthetic fixture. In implementations of the athlete technology, the pressure sensor is placed at a sensor placement site on top of a muscle head to be measured. Much like placing the Laplacian electrode directly on top of the innervation zone for maximizing dynamic range of the signal, placing the FMG sensor on the muscle head tends to maximize the dynamic range of measured force produced by that muscle. In addition, a mechanomyogram can be recorded from the force myography sensor. The mechanomyogram is a signal representing the internal vibrations of the muscle tissue and can provide additional athlete data for determining fatigue. In some implementations, the IMU's internal accelerometer may be used to record the mechanomyogram.

The combination of measuring the sEMG at the innervation zone and the force at the muscle head provides a powerful technique for characterizing, for example, the electromechanical delay (EMD) of the muscle. The EMD is the time it takes for a recruitment signal to generate a force. Although relying on EMD to infer muscle state can be problematic when the electrodes are not properly placed at the innervation zones, this concern is addressed by algorithmically locating the innervation zones and muscle heads where the electromyography and force myography sensors are to be placed, respectively. This ensures the accurate placement of the Laplacian electrodes and force myography sensors in an athlete garment tailored to the athlete participant's body.

Using signals from the innervation zones and from the muscle heads together can provide new and useful information. Among a wide variety of other uses, for example, the amplitude of the innervation zone signal can be compared with the FMG signal to measure one or more athlete performance factors such as fatigue, and to measure the EMD and spectral shifts in the innervation zone signal.

Because each pressure sensor will always be under pressure when the athlete garment holding the pressure sensor against the skin of the athlete participant is worn in the normal resting state, it is possible to measure a degree of muscle lengthening at times when muscle lengthening causes the muscle head to pull away from the fabric of the garment. Such a measurement can be a proxy for flexibility of the muscle and provide insight when combined with other athlete data (such as kinematic data) for quantifying a degree of overextension of the muscle.

In some embodiments of the athlete technology, the pressure sensor can be made in a variety of ways. In some implementations, the pressure sensor can be a capacitive, multilayered fabric pressure sensor. In some examples, the pressure sensor can be formed from a piezo-resistive foam.

Pressure Sensors

Some implementations of the athlete technology use capacitive pressure sensors configured as a sandwich of two PEDOT:PSS coated polyamide fabric circles with the middle of the sandwich being a polydimethylsiloxane (PDMS) dielectric. The sandwich is sewn together using non-conductive thread. When the muscle pushes against the fabric of the garment, the capacitance of the corresponding sensor changes and is measured as an indication of the muscle expansion.

In some implementations that use piezoresistive foam, the foam is fabricated using a melamine sponge dipped in an aqueous PEDOT:PSS solution. In some examples, a conductive polymer can be mixed with a dissolvable element such as sugar crystal. The mixture is placed in a solvent, removing the sugar crystals, leaving behind a porous structure. Two conductive threads are placed at opposite ends of the sponge and then the sponge is encapsulated in a sealing material to protect it from the elements. This pressure sensor is placed between the compression fabric of the garment and the muscle. When the foam is compressed by the force of the muscle, more conductive paths are created, decreasing the resistivity of the foam. The resistivity is measured as an indication of the expansion of the muscle.

Customizing the Sensor Placement Sites and Garment Fit

As discussed earlier, in some implementations of the athlete technology, the selection of sensor placement sites is carefully made to match the locations of features of the musculoskeletal structure of each athlete participant, such as innervation zones and muscle heads. The locations of those features are unique to individual athlete participants. In some implementations, the sensors are not physically attached to the skin of the athlete participant but rather are placed against or in proximity to the skin of the athlete participant by an athlete garment worn by the athlete participant and to which the sensors are attached or in which the sensors are incorporated or both. The placement of the sensors with respect to the athlete garment is customized to reflect the unique locations of those muscle features on the intended athlete participant. In addition, for the sensors to be situated in the proper sensor placement sites when the garment is worn and for the garment also to be comfortable and not to impede the free motion of the athlete participant, the athlete garment is designed and fitted (that is, custom designed and fitted) to the athlete participant's body appropriately. In other words, one or more custom athlete garments having custom sensors in custom sensor placement sites must be created for each athlete participant.

All of the sensors and multiple units of the sensors described throughout this document may be attached at appropriate locations to an athlete garment for this purpose.

The needs of athlete participants with respect to the athlete garments to be created vary greatly depending on the athlete activity and on their positions or roles with respect to the athlete activity. The form and structure of an athlete participant's body also depends on positions or roles even within an athlete activity (e.g., a sport), where certain playing positions demand specific physiology. For this reason, professional athlete participants, for example, commonly wear professionally tailored jerseys or other garments to ensure a good fit for comfort and performance. Similar considerations apply to the athlete garments and customized athlete garments to be used as part of the athlete technology. In some implementations of the athlete technology, the sensor placement sites are determined precisely based also on a range of other physiological parameters.

In some implementations of the athlete technology, making the customized garment or garments for a particular athlete participant involves measuring a variety of parameters including, for example, the shape and size of the athlete participant's body and parts and body segments of the athlete body including joint locations and joint axes, locations at which the skin is stretchy or not stretchy, and ranges of motions of different parts and segments of the musculoskeletal system. In some implementations, determinations are made of where, beneath the skin, the muscles are highly innervated. A calibration process described below helps to ensure that the correct sensor placement sites are identified and applied in designing and making the garments.

In some cases, for high definition sEMG sensing, a tight grid of sEMG sensors could be used.

Once finished, the new custom-fitted athlete garment is donned by the athlete participant to verify sensor locations and to create a baseline of athlete data from the sensors. After the baseline of athlete data is created, the athlete participant's garment and personal mobile device are registered with a communication hub and network to begin active use of the athlete technology.

Compressed Sensing

Compressed sensing is a signal processing paradigm that allows signal sampling and subsequent reconstruction of a sparse or near-sparse signal by randomly sensing it. The average sampling rate ends may be far less than typical Nyquist-rate sampling. Compressed sensing at the athlete garments allows the athlete garments to save energy on the transmission of data to a communication hub. Compressed sensing relies on the signal of interest to be sparse in some domain.

To model a signal, signal samples are collected in a time window in the n-dimensional vector x∈Rⁿ and its representation with respect to vectors is made to be columns of a matrix S. S is considered a base and then x=Sξ with ξ=S⁻¹x. x is k-sparse if ξ has at most k non-zero elements and k<<n.

When this happens, the true number of degrees of freedom of x is less than n. The signal information is then represented by the measurement vector y∈R^{m} which is constructed by projections to a set of sensing vectors arranged as rows of the sensing matrix A.

y=Ax=ASξ

Often m<n measurements are enough to retain most of the signal's information and the compression ratio of the measured signal then becomes n/m.

When the matrix AS fulfills certain conditions, and x can be recovered from y even though A is a dimensionality reduction.

However, this is a general solution and the athlete technology uses the signal's statistics as a priori information to design a sensing matrix to maximize collection of a signal's energy and therefore maximize information. Additionally, because the contact impedances of an athlete's electrodes will change from session to session due to varying sweat and sebum levels, the athlete technology uses a novel calibration method. The change in contact impedance changes the frequencies that are measured and the skin-electrode interface acts in essence as a low-pass filter to varying extents. The sensing matrix adapts to measure the altered signal by first sampling the signal at the Nyquist rate and identifying the structure and statistics of the signal, and creates a rakeness-based sensing matrix to maximize energy collection of that signal.

There are five sections to the signal chain shown in FIG. 26 (see https://ieeexplore.ieee.org/document/7307256.) The first stage is acquisition (low noise amplifier, analog front end) which simply interfaces with the electrode. Next the signal is sent to stage 2, discretization. The signal then passes through two routes, the compression stage (step 5) and the analyzing stage (step 3). Upon starting an exercise session, the analyzing stage is activated and sends raw Nyquist measurements of the signal to the user's device, or to the hub, or the raw Nyquist measurements are analyzed using machine learning models running on a processor in the athlete garment. A sensing matrix is created on-device or in the cloud and is returned to the athlete garment to overwrite the compression stage's measurement matrix in the MAC register. The compression stage encodes the samples into compressive measurements using the updated matrix and are then sent back to the device or hub.

Design of the sensing matrix involves a tradeoff between a broad solution that will work acceptably but will not rely on the a priori information of the signal being measured, or an overly specific solution in which any atypical measurements will be inaccurate.

After the signal is amplified through the analog front end (AFE) and discretized by the analog-digital converter, the projections are performed by digital multiply and accumulate (MAC) operations.

The athlete technology reduces the complexity of the measurement circuitry by using multiplier elements when possible and constructs the measurement matrix as a ternary sensing matrix. This stores the matrix as 1s, −1s, and 0s. When the matrix contains a 0, there is no MAC operation and this saves measurement energy

FIG. 25 illustrates an example of placement of sensors, wires, and other components on an athlete garment and the locations of the components relative to body segments and body parts of an athlete participant. In the figure, the following numerals identify the indicated components:
1. Inertial Measurement Unit (IMU)
2. Haptic feedback unit
3. Laplacian sEMG electrode
4. Force myography (FMG), mechanomyography (MMG) sensor
5. Insulated sinusoidal wire
6. NIRS device
7. Circuitry housing including CS, FMG, IMU
8. Transmission horseshoe shaped wire
9. Physical connection snaps
10. Outer active TEB electrodes
11. Inner passive TEB & ECG electrodes
12. Wireless module and circuitry housing
13. Speakers for auditory feedback

Photogrammetry

In some implementations of the athlete technology, one of the inputs to the biomechanical model (discussed below) is a 3D scan of each athlete participant. From the 3D scan, the technology can calculate the mass of each body segment of the athlete body, the center of mass of each body segment, and the muscle geometry and joint locations for each body segment. The 3D scan is generated using photogrammetry in which multiple photographs are taken around the athlete participant, and the photographs are stitched together based on features common to the photographs. The common features are saved as points of a dense point cloud in a 3D space. Extraneous points are removed manually from the cloud and the remaining points are connected as triangles, creating a rough 3D mesh. Smoothing and interpolation are applied between the points in the 3D mesh to reduce its complexity. Information about this technique is explained in http://www.gancell.com/reports/Final_Basics_Photogrammetry.pdf, and incorporated here by reference.

### Calibration Procedure

In some implementations, the calibration procedure can be done in four stages as follows:

The first stage locates the innervation zones using one of n different sizes of mid-calf compression leggings. The athlete participant is evaluated to identify one of these sizes to be used. Inside the compression leggings are patches of hook-and-loop attachment surfaces located at regions of likely innervation zones. HD-sEMG panels (each bearing a grid of sEMG sensors) are attached to these regions. This is done using latching hooks affixed to the backs of the panels for latching to loops on the inner attachment surfaces located at the regions of likely innervation zones. To increase comfort, the attachment of panels and the corresponding collection of signals may be done in stages, with a limited number of muscle groups measured for each stage. After each stage, the leggings would be refitted with the HD-sEMG panels moved to new locations. The athlete participant performs exercises for each stage to collect muscle activation data. If data collection needs to be improved for a muscle, a readjustment may need to be made of the locations of the panels. Dynamic and isometric exercises are included in this process to provide a range of spatial signal characteristics and accommodate adjustment for non-stationary innervation zones. Afterward, the corners or edges of the HD-sEMG panels are marked on the outer surface of the athlete garment and are used as a measurement frame.

In some implementations, a set of n sizes of leggings are each fabricated to have a conductive wire structure and a grid of electrodes is integrated into the leggings using the same manufacturing process used for the Laplacian electrodes. The conductive wires pertaining to a particular grid of electrodes converge to a connector located in or near the waistband of the calibration leggings.

This configuration would have the measurement panels outlined on the surface of the garment and each measurement panel's measurement frame would be recorded in the second stage. The athlete participant would remain in their calibration garment.

The second stage is a 3D body scan of the athlete participant. The athlete participant enters the scanning space of a commercially available body scanning system. The scan creates a 3D, volumetric model of the athlete participant with sufficient resolution to locate relevant surface anatomical landmarks. The athlete participant assumes multiple “extreme” pose” to allow for measurement of volume and perimeter changes. For example, in one pose the athlete may stand up straight with their arms out to their sides or in front of them, or in another pose with their legs spread apart, standing up straight, with their arms lifted straight into the air. Some positions will allow muscle bellies to be more accentuated improving the placement of force sensitive resistors.

The third stage is a data manipulation stage. The data manipulation enables the athlete technology to locate anatomical landmarks, compute volumes of body segments, design the Laplacian electrode, compute the placement of sensors, route the conductive polymer wires, and design clothing panels, among other things.

From the 3D volume, section planes are extracted at intervals of 0.5 cm. A convex hull is computed for every continuous section, and the length of the convex hull curve is saved. Using these convex hull curves that define the exterior boundaries of the body in combination with a surface map describing surface deformation, a routing pattern is computed. Sinusoidal or horseshoe patterned wires are projected to a vertical array of convex hulls along routes to balance certain constraints, including one or more of the following: minimal surface deformation or motion (thereby increasing comfort), minimal wire length (maximizing SNR), minimal anticipated athlete garment travel along the surface of the skin to maintain sensor locations (maximizing data accuracy), and creation of an aesthetically pleasing route, and combinations of these constraints.

Using a surface map of known comfortable pressure across the surface of the body (obtained from literature) and the material properties of the fabric and wires, section perimeters of the planned athlete garment are calculated. The section perimeters can enable the determination of optimal compression and range of motion where needed. Seam locations are placed to maximize comfort and ease of motion across the calibrated range of motion, such as muscle to muscle interfaces, for example the posterior deltoid and tricep. Once the seams are projected to the planned athlete garment, the fabric panels are extracted as 2D shapes. These 2D panels retain the planned paths of the wires and the sensor locations. These paths and locations are exported as a cartesian sequence of points for the fabric printer to utilize.

After this third stage, enough data has been gathered to fabricate the athlete garment as described later.

The fourth stage is validation and verification of the created athlete garments.

Errors for every sensor location across a set of athlete garments (for example, for a team) are recorded as a distribution. This data is incorporated into the calibration model to improve the design of future batches of athlete garments. Athlete garments with fit or sensor placement error above a threshold are remade. Athlete participants don their new clothing and perform a maximum VO2 test by increasing speed on a stationary bike every 4 minutes until exhaustion. During this exercise they are also hooked up to a respiratory machine that measures their lung volumes. This respiration data is trained to their impedance measurements so a better calculation of lung parameters can be achieved. The cycling exercise collects a dynamic range of NIRS (near infrared spectrocsopy), sEMG, and pressure readings, which will allow for more precise oxygen and mechanical dynamics from the start.

In some implementations, the calibration procedure can be designed to enable a consumer to perform it on an athlete garment received directly (for example, at home) by the athlete participant. Among other features designed to achieve this capability are scalable manufacturing methods including inkjet printing of conductive elements and 2D shape laser cutting, and shipping of calibration garments to the home where a mobile phone app could be used to perform the photogrammetric scan.

Body Segment Lengths, Body Segment Convex Hulls

The photogrammetry technique can be applied for varied poses by the athlete participant to facilitate determinations about where in an athlete garment to include fabric that will provide “give”, and where to apply ideal shapes to minimize fabric stitched boundary shear.

Providing a textured, irregular graphic pattern on the athlete garment helps the camera stitch the captured frames together to form an accurate 3D model of the athlete participant.

As shown in FIG. 20, in some implementations of the athlete technology, after the 3D mesh pattern is marked, the athlete participant assumes selected poses. In some cases, the poses are extreme conformations of the athlete participant's body in the sense that their arms, legs, and torso are at extreme ends of the ranges of motion, for example, arms hanging down to the side and held straight up; standing up tall; and standing with feet spread apart. The poses are captured in still images and analyzed manually. The purpose of these poses is to enable identification of locations in the custom garment that must accommodate greater ranges of motion and volumetric changes in body segments. For example, in the underarm and groin areas extra panels of fabric may be needed to permit greater athlete participant mobility.

Biomechanical Modeling

Biomechanical modeling (BMM) typically involves creating a computer model of the musculoskeletal system using physiological properties, such as muscle and bone density, tendon elasticity, tensile strength of tissues, and sometimes scaled volumes and shapes of various structures. The physiological properties taken into account in a biomechanical model depend on the behavior one is interested in modeling, for example, kinematics, injury risk, or kinetic efficiency. In implementations of the athlete technology, a variety of physiological properties can be taken into account, including properties determined empirically to be relevant to the goal of the modeling based on the goals of the athlete technology.

Ideally, a biomechanical model would take account of properties and behavior of every muscle in the athlete participant's body, but making measurements for every muscle is not yet feasible. It's common practice to group similarly-acting muscles together to reduce complexity of the model. Biomechanical models vary in the extent to which they group muscles. The more muscles that can be measured and incorporated in the model, the more accurate and highly resolved are the results produced by the model. An example of common biomechanical modeling software is described at https://www.ncbi.nlm.nih.gov/pmc/articles/PMC4397580/, incorporated here by reference.

Typically the biomechanical model uses empirical data captured in a laboratory setting by a video camera from people wearing visible markers at selected marker sites on their body (or on a motion capture suit). In addition to static poses, athlete motion can be captured by the video camera, which enables additional information to be derived such as approximate joint loads or approximate muscle contributions to the motion at successive times during a body motion.

In some implementations, the athlete technology can supplement the musculoskeletal system's natural feedback loop of sensing muscle state and actuating muscles based on the sensed state. The technology's sensors include IMUs, sEMG, and FMG sensors as discussed above. The actuations of muscles within the loop are constrained by the musculoskeletal system properties as reflected in the 3D scan of the athlete participant.

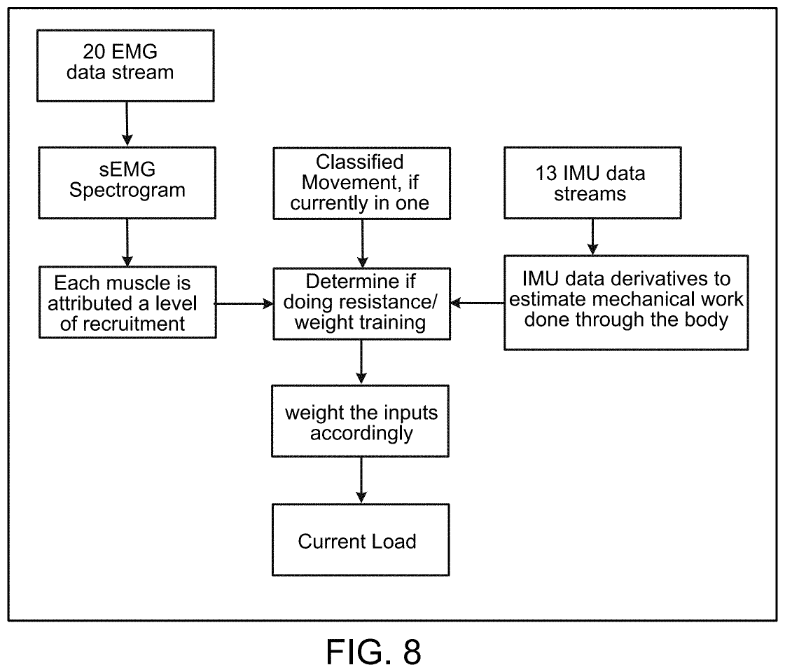

In some implementations of the athlete technology, a selection of muscles and their activity (recruitment and resulting actuation) and the body motions resulting from the combination of muscles' activities are measured (see FIG. 8). The sEMG electrodes are placed at sensor placement sites at the centers of the innervation zones to measure recruitment electrical signals. The FMG pressure sensors are placed at sensor placement sites at the muscle heads to measure muscle actuations. The IMUs are placed on segments of the body segment at sensor placement sites nearest to the centers of mass of the respective segments. The muscles selected for measurement are not the only muscles included in the biomechanical model, but they provide empirical data that help the other muscles of the model adjust, making for a computationally lighter biomechanical model. During a muscle contraction, the fibers within the muscle pull against plates in the fiber, bringing the plates closer together, shortening the fiber's length. However, in conserving the volume of the fiber, the fiber thickens, producing the familiar bulge. The peak of this resultant curvature is called the muscle head. By combining a photogrammetric model, and signals from IMUs, Laplacian electrode sEMG signals, and force myography information, portions of the musculoskeletal system and certain athlete performance factors can be effectively characterized.

Additional information about the athlete technology is illustrated in FIG. 9 (an overall functional and structural block diagram of the athlete technology incorporating features described above and others, FIG. 10 (a block diagram of a process for determining load to body response ratio in the athlete technology, FIG. 11 (a block diagram of a process for determining real-time risk of injury based on a load to body response ratio in the athlete technology, FIG. 12 (a block diagram of a process for determining fatigue level based on a current body state score and a conditioning score in the athlete technology), FIG. 13 (a block diagram of a process for determining a high conditioning score, FIG. 14 (a block diagram of a process for identifying trends to understand the impacts training load/type has on a players risk to injury), FIG. 16 (a block diagram of a process to determine a body state of an athlete participant), and FIG. 17 (a block diagram of a portion of analysis steps performed by the athlete technology.

### Fabricating Waterproof Electronic Textiles Using Polysiloxane Coated Conductive PEDOT:PSS Ink Wires on Polyester Fabrics

Due to its material properties and economic viability, polyester (PET) fabrics are a commonly used fabric for making active-wear garments. However, material characteristics of PET fabrics may not be appropriate for use in electronic textiles (e-textiles). It is possible to improve PET fabric conductive properties while simultaneously maintaining the ability to be rubbed and washed. As shown in FIG. 15, a process to achieve these qualities is to repeatedly inkjet print poly(3,4ethylenedioxythiophene):poly(styrenesulfonate) (PEDOT:PSS) solutions containing 4-7 weight percent dimethyl sulfoxide (DMSO), in a sinusoidal or horseshoe wave pattern, on to the PET fabric. PET fabrics, with conductive ink wires, are made specifically for each individual athlete participant to optimize comfort and sensor measurement accuracy and fidelity. To allow the fabric to be washed another inkjet printer is used to coat the PET fabric with hydrophobic polysiloxane over the same PEDOT:PSS pattern. Then the custom PET fabric panels are woven into tailored athlete garments.

The conductive PEDOT:PSS solution used in the inkjet printing is obtained by adding 4-7 weighted percent DMSO to the PEDOT:PSS solution, and then sonicating for 30-45 min in an ice-water bath. Sonication uses sound energy to speed up the dissolution of DMSO into the PEDOT:PSS.

The PET fabric is prepared for inkjet printing by multiple ethylene oxide and deionized water rinses, multiple dryings in an oven to constant weight, and air-plasma atmospheric treatment.

The optimal PEDOT:PSS solution inkjet printing coordinates are determined using athlete data specific to each athlete participant with other hardware locations as constraints. The PEDOT:PSS solution is inkjet printed onto the fabric in a sinusoidal or horseshoe at an angle of 45° wave pattern to minimize resistance and axial stretch. The inkjet head repeats printing the solution multiple times to make sure enough of the solution is present on the fabric.

The conductive PEDOT:PSS coated PET fabric is then placed into a vacuum dryer for 30-45 minutes to dry. After the conductive PET fabric is dried it must go through another inkjet printing cycle to become waterproof. The inkjet printer can contain two types of polysiloxane dispersions dimethyl polysiloxane, or methylaminosiloxane with glycidyl trimethylammonium chloride. The hydrophobic polysiloxane is printed over the same PEDOT:PSS conductive patterns that were previously created.

The PET fabric panels with inkjet printed and coated wires are now ready to be woven together to form custom tailored athlete garments using known manufacturing processes.

Interaction of the Athlete Participant with the Garment

In some implementations of the athlete technology, a variety of user interface techniques and combinations of them can be used to provide feedback to an athlete participant based on athlete performance factors determined as a result of sensing activities. The user interface techniques can include visual, tactile, auditory, and others, and combinations of them.

Visual Feedback

In some examples, as shown in FIG. 24, a device worn on the forearm of an athlete could display an “energy meter” using a colored stack of bars such as 5 green bars, 4 yellow bars, and 3 red bars. The bars would be used to indicate, based on computations of the athlete technology, fatigue and extent to which the athlete should be considered fatigued or not fatigued.

In some implementations, this user interface feedback mechanism is tangible and always at hand. This user interface device in the form of a bar or energy meter indicates to the athlete participant how fatigued they currently are, their current risk of injury, and how important it is to be cognizant of their fatigue level.

In some examples, the bar or energy meter is located on the left or right middle forearm, depending on the athlete participant's preference. The number and colors of the LEDs in each of three groupings helps enhance for the athlete participant the apparent importance of each subsequent LED “lost” from the state of full energy. When all of the LEDs and the bars are lit, the athlete participant will understand that they are not fatigued at all. As their fatigue level increases, the LEDs turn off sequentially from top to bottom. In the green grouping, each LED contributes to 20% of that grouping, in the yellow, 25%, in the red, 33%. The increasing contribution of each colored LED to its group provides feedback indicating an increasing cost of losing it. In tandem with the total number of LEDs lit (8 out of 12, for example), the athlete sees, for example, they are on their last green bar, nudging them to think more about their state. The correlation of the lighting of the LEDs with physiological factors can be determined empirically for each of the athlete participants. By learning the athlete participant's dynamic range of exertion and physiological state in combination with population level insight, the athlete technology can control the lighting of LEDs accordingly.

The location on the body most sensitive to passive physical feedback is the hands, with sensitivity dropping off beyond the middle forearm. Placing a haptic feedback device in the middle forearm narrows the range of locations the athlete participant needs to pay attention to in their mental schema of their body. Moments natural to provide haptic feedback could include transitions from green to yellow or yellow to red, or from one yellow LED to the next and so on. In some cases, the athlete participant will be able to sense the acceleration of their fatigue as it develops.

By the use of such a user interface feedback device, the athlete technology can ensure low latency and high performance of providing feedback to athlete participants. To achieve this, in some implementations, the athlete technology pushes computations required for feedback closer to the edge of the local network. In some cases, the athlete technology can deploy a cloudlet (e.g., a hub) to provide computational resources near the sensors on or near the athlete garment and a common connection or access point for the athlete garment. As discussed above, computation using the compressed sensing data can be dealt with on the athlete garment. In instances when the compressively sensed data is not reconstructed before machine learning takes place, the application of machine learning would be in a compressed learning mode.

In some instances, haptic feedback can be used by placing one or more haptic devices in an athlete garment. The haptic feedback could be delivered to the surface of the skin at a part of the body that corresponds to information generated by the athlete technology. For example, if the athlete technology determined that the athlete's forearm was being moved beyond the normal bubble of motion predetermined by the athlete technology as typical for the given athlete participant, haptic feedback to be provided to that forearm through a haptic device in the athlete garment.

In some implementations, the inside or outside surface of the athlete garment may be inkjet or screen printed with a pattern that visually guides the athlete in putting the compression garment on in the correct way. For example a visible line may be printed to match the curve of the tibia or circles printed to match over ostial protuberances such as the knee or hip. For example, if the compression athlete garment prevents the sensors from moving but the fabric is rotated around the body segment, data from the sensors may be less accurate.

In some cases, auditory feedback can be provided to the athlete participant including spoken feedback, noises, or combinations of them interpretable by the athlete participant as indicating a wide variety of feedback information.

In some implementations, Bluetooth headphones may be included in the athlete technology. The headphones can connect to an athlete participant's user device for receiving auditory feedback from a digital assistant or a coach.

In some implementations, speakers may be integrated into the garment, for example, in the supraclavicular fossa, the region above the clavicle. This directs the sound towards the athlete's ears and allows for situational awareness.

Body Sensor Network

In some implementations, the sensors attached to the athlete garment and used to instrument the body of the athlete participant can be organized and interconnected in a communication network that is wired or wireless or a combination of the two. For this purpose, a network hub (or cloudlet) that manages the operation of the body sensor network can be mounted on the athlete garment or externally from the athlete garment or a combination of the two.

In some cases, the sensors attached to the athlete garment and used to instrument the body of the athlete participant can participate in a network organized and managed by an external device. The sensors can be programmed to search for permissioned external networks exposed by user devices or cloudlets. Data collected at each sensor can be streamed in real time through the communication network to other devices that are part of the athlete technology. In some cases, the collected data can be stored temporarily in memory associated with each of the sensors or in memory associated with the on-garment hub.

When multiple athlete garments are worn, inter-garment communication connections can be made. This reduces the number of wireless radios needed, reduces total power consumption of worn athlete garments, and allows on-garment hubs to be more specialized in their form and function. For example, an upper athlete garment can focus on wireless communication and delivering athlete insight, whereas a lower athlete garment can process more sensors or information.

User Devices

In some implementations of the athlete technology, mobile devices of athlete participants can create a Wi-Fi network allowing the athlete garment worn by the athlete participant to stream athlete data. The athlete technology can include CS (compressively sensed) data storage and the CS data can be reconstructed. A Huffman encoded deep neural net (DNN) can be used for predictions on CS reconstruction.

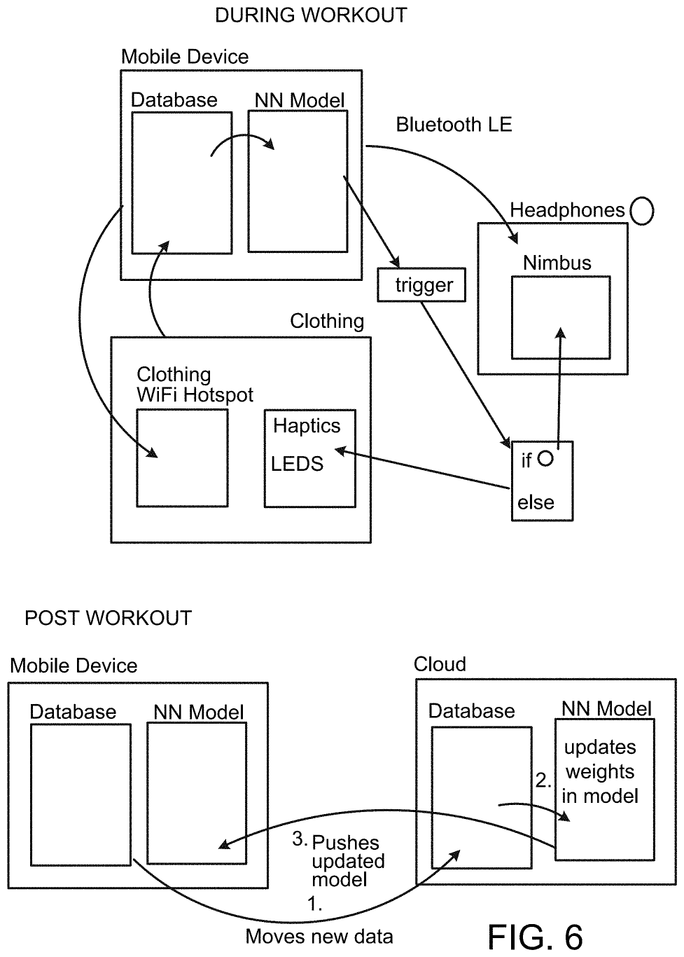

As shown in FIG. 6, in some cases, when an athlete participant begins a new athlete activity session, a new model can be downloaded to the network for use in processing data acquired during the new athlete activity.

After the session ends, the newly acquired data can be uploaded to a cloud server. The upload can include the raw CS data and any commands or suggestions sent to the athlete participant during the session.

Cloudlet

In some cases a local cloudlet can be used to broadcast (expose) a hidden Wi-Fi network for CS (compressively sensed) data reception. Local graphics processing units can be provided for multiple athlete participants for CS biosignal reconstruction. In some cases, the cloudlet can provide the same quality of service level as would be provided from the cloud server. The cloudlet can send data visualizations to participant stakeholders directly through the local network.

Cloud

In some instances, the central cloud server can manage CS data reception and CS reconstruction. The cloud can be in charge of updating a personal DNN using new personal data and new population level data, and Huffman encoding the DNN and send it back to the local network when an app running on one of the athlete participant's mobile device opens to begin a new session.

In some implementations, to make effective use of mobile devices in the form of smart phones, the athlete technology uses Bluetooth and Wi-Fi signal bands and their compatible protocols, such as 802.11.

The athlete technology is designed to provide adaptive packet behavior. Because the process of confirming packet reception is energy intensive, the athlete technology determines the packet dropout percentage compared to RSSI and operates as if it were randomly sensing 80-100% of the sent packets. As a result of losing information that the athlete technology had planned on using to reconstruct the signal to an appropriate level, the athlete technology is designed to use a rakeness-based sensing matrix that is slightly liberal in its sampling so to compensate for information loss.

### Teams

Athlete activities such as team sports happen everywhere. A team, for example, may not play or practice in the same place every time. A framework for computational support on a home venue can be achieved fairly simply using a server rack and a wireless access point for connecting the server to sensors and network hubs on athlete garments or network hubs located on the home field. However, when a team travels to other venues, the supporting infrastructure must be movable to enable communication with the athlete garments. Connecting to Internet access points that are not self-managed may create a security risk. In some cases there won't be a secure wired or wireless Internet access point available. The cloudlet approach yields faster delivery of information and can be used to ensure privacy and ownership of data streams generated by athlete participants. The cloudlet can be implemented using any combination of cellular, LTE, WiMax, and 5G communication, and combinations of them, for example.

For traveling groups or teams of athlete participants, a portable cloudlet can be implemented as an aesthetically pleasing and compact portable server rack. The portable cloudlet can enable, for example, sports teams and their management to have a simple, consistent, dependable, and adaptable experience. It can be thought of as providing “n % up-time” for a number of parameters. The portable cloudlet facilitates the analysis of sport teams of athlete participants wearing athlete garments.

In some implementations, three scenarios imply the functionality to be included in the cloudlet. One scenario is when the cloudlet is hardwired and has full speed access to the cloud. The second scenario is when the cloudlet must rely on its own wireless bandwidth and may have a limited speed connection to the cloud. The third scenario is when there is no connection to the cloud. In the third scenario, the cloudlet can provide a minimum level of predictable analytics to the team, including risk of injury (RoI) and exertion levels. In that scenario the cloudlet processes some or all athlete data necessary for those purposes at a location closer to the team, thereby reducing the delay of notifications sent to the athlete participants and athlete stakeholders such as coaches.

The reduction in delay is important because athlete participants tend to have a brief window of time of sensorial memory. Depending on the sense (touch/haptic, sight, auditory, olfactory) memories are retained for a varying small amount of time. It is important for teachable moments to be addressed soon after they happen so that more effective learning and adaptation can occur. This holds true for the athlete participant correcting their form, or for a perceptive coach or trainer or other athlete stakeholder connecting their intuition with real time information of their choice on their user devices. Depending on the physiological indicator or team parameter, the update interval or frequency required to provide adequate utility will differ. To provide this to the team, computation resources will be split optimally between the athlete garment processor, cloudlet, and a separate public or private server (e.g., the cloud). As the machine learning model learns from the team's data, better prediction can be achieved in the optimal setting and more indicators or better prediction can be provided in the worst case scenario. Within some boundaries, teams can choose what data visualization and prediction parameters matter to them.

In some examples, the athlete technology includes four main components. In the literature, an on-body system of heterogeneous sensors is called a WBAN. The various sensors can be hardwired, or wirelessly connected to a sink node, which collects packets of data from the wireless on body sensors and transmits the data to a mobile device or other access point. In some implementations of the athlete technology, the athlete garments will not serve as a wireless hub to additional on-body sensors; in some implementations, the athlete garments will serve as a wireless hub.

Multiple athlete garments can be connected to the cloudlet using a Wi-Fi link operating on the 802.11 protocol. The packet transmission protocol can be UDP. Significantly, due to the rakeness property of the compressed sensing, information from the athlete's body is captured in spikes or waves over time, depending on the signal statistics at any moment during measurement. As the machine learning models learn patterns in the athlete data, network optimization can be used to predict packet loss and packet rate, and account for them to reduce delay and improve the machine learning model's robustness to loss of athlete data.

### Modes of Operation

Implementations of the athlete technology can operate in a variety of modes with respect to the athlete participants and athlete stakeholders participating in athlete activities, the context of the athlete activities, and the nature of the athlete activities, among other things. The technology controls access by athlete participant and athlete stakeholders to various features of the technology by enforcing a permissioning system for use and access. The permissions can differ for different modes and athlete participants.

In some modes, a team of athlete participants may split into groups for different athlete activities. One exercise physiologist can view the athlete activities of one group while another exercise physiologist can view the activities of the second group.

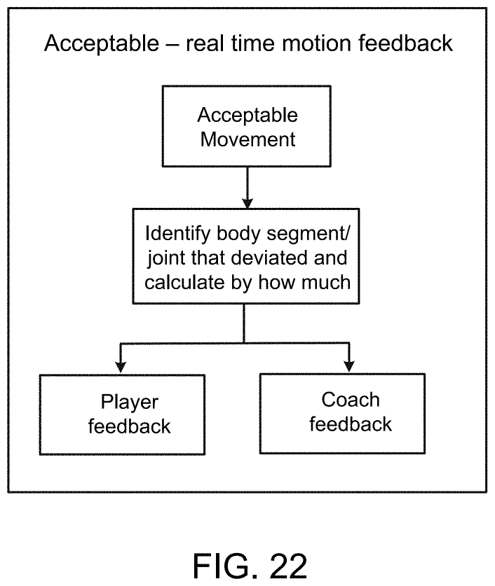

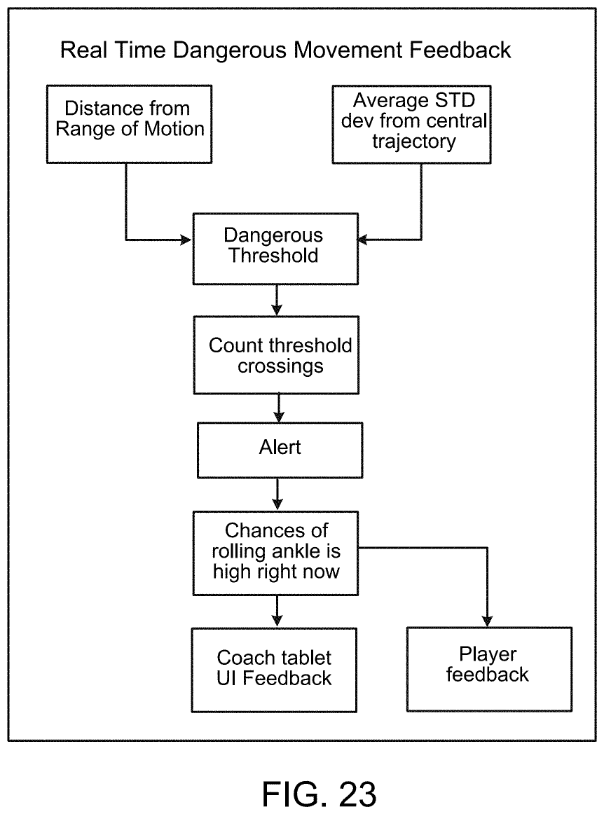

As shown in FIGS. 22 and 23, in some modes an athlete participant engages in an athlete activity (for example, a workout) individually. In such a single-user mode, data is sent to a personal device of the athlete participant (such as a mobile phone). In some modes the athlete participant engages in one-on-one training with a trainer or coach. In such sessions feedback is also or instead sent to a device controlled by the trainer or coach.

### CS Reconstruction and Analytics

When athlete participants train or engage in other athlete activities alone or one-on-one with a trainer or coach, the communication hub may be inactive or out of range of the athlete garment worn by the athlete participant. Since the hub's wireless network cannot be found, the athlete's user device creates its own WiFi network to which a single athlete garment can connect.

When the mobile application provided as part of the athlete technology is opened on the athlete participant's device, the specific athlete activity prediction model for that athlete participant is automatically downloaded or updated in the background. This specific model is based on their historical exercise activity data and on up-to-date insights based on exercise data for relevant populations of athlete participants. The model is Huffman encoded to a smaller size in the cloud before being downloaded.

Compressively sensed (CS) data can be reconstituted on the athlete garment or can be transmitted from the athlete garment to the athlete participant's device to be reconstituted in real-time, or a combination of the two, and the reconstituted sensed data can be applied to the model as received. The athlete participant's device may or may not pair with Bluetooth earbuds to enable Nimbus' voice feedback. If Nimbus is not enabled, the user experience (UX) adapts to lean more heavily on other feedback mechanisms like a visual wrist meter or arm meter (as described above) and haptic feedback (as described above) and will reduce auditory feedback to tones or beeps. During this athlete activity session, the raw compressively sensed data and the feedback and insight data provided to the athlete participant are saved. Once the activity ends, these data are uploaded to the database at the cloud server hosted by the athlete technology. The compressively sensed data is reconstructed in the cloud. A significant feature of the athlete technology is that the athlete insights provided to the athlete participant can be compared to subsequent athlete performance factors to quantify the influence of the athlete insight feedback on the athlete performance factors. This self-check, along with the compressively sensed data, is added to the athlete participant model's data pool, and the neural network's weights are updated.

In some implementations of the athlete technology, when a trainer's mobile device connects to the hub, the user interface for the trainer is tailored for their role and interaction with the athlete participant.

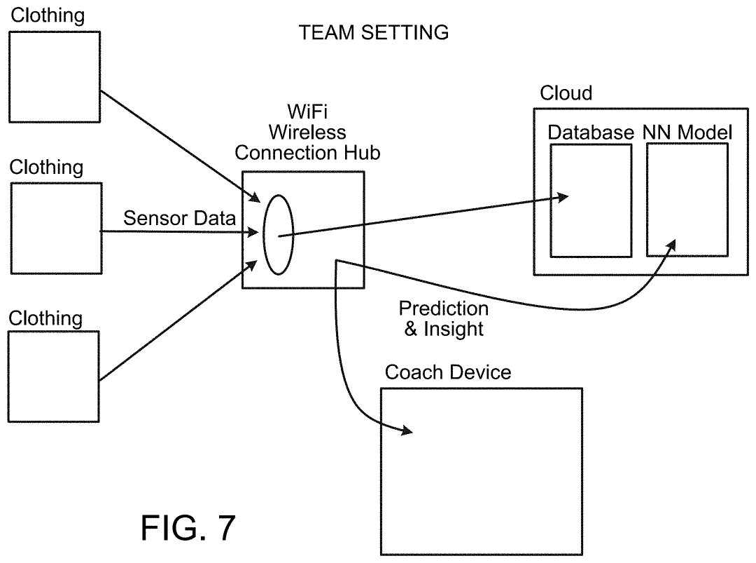

As shown in FIG. 7, in some examples of a team mode of operation of the athlete technology, athlete garments of the athlete participants who belong to the team all connect to the hub's accessible Wi-Fi network.

In a team practice mode, upon boot-up of each athlete participant's athlete garment, the athlete garment searches for and connects to the WiFi network accessible from the hub. Each athlete participant's compressively sensed data is wirelessly transmitted from their athlete garment to this hub where it is reconstructed. Because the hub may be moved to wherever the team may practice, the athlete participant may not have WiFi access or sufficient speed to provide the quality of service (QoS) needed. Quality of service is the ability to guarantee a certain level of performance for a data flow. To address this, the hub can store the athlete activity prediction model for every athlete participant, along with a team dynamics model which models playing patterns of the athlete participants of the team, locally on the hub.

In some implementations of the athlete technology, the quality of service will be sufficiently high that data from athlete sensors and athlete motion insights will appear essentially instantaneously on the user interface of the athlete participant's device as the athlete participant is engaging in athlete activity.

In some examples of team activity, during basketball practice, 5v5 scrimmages are common. The remaining five benched players are swapped in during play to discover optimal team dynamics. In such a context, the hub must be able to process data streams of athlete sensors of fifteen athlete participants in near real time.

To do this, the deep neural networks (that is, the biomechanical and physiological models) for all athlete participants and the team are encoded (using, for example, Huffman coding) to a smaller size by a processor running on a server on the cloud platform, and then downloaded to the hub. The coding reduces complexity of the networks and the energy needed to execute them locally.

The hub remains connected to the cloud platform when not actively in use, effectively dividing processing and storage activities between the hub and the cloud platform to maximize quality of service.

Coaches, trainers, exercise physiologists, managers, and other athlete stakeholders can have tablets or other devices connected to the hub's WiFi network. Different athlete stakeholders can be permitted to have predefined different levels of access to the sensor data and athlete motion insights of respective athlete participants and teams. Every device and every athlete stakeholder is permissioned to manage when, where, and from whom sensor data and athlete motion insights can be accessed.

Other implementations are also within the scope of the following claims.

1. A method comprising
receiving athlete data from one or more inertial measurement units associated with one or more muscles of an athlete engaging in athlete activity,
receiving athlete data from one or more Laplacian electrodes associated with the one or more muscles of the athlete and indicative of sEMG signals,
receiving athlete data from one or more pressure sensors indicative of force imparted by muscle heads of the one or more muscles, and
characterizing one or more athlete performance factors of the athlete associated with the one or more muscles based on the athlete data from the one or more inertial measurement units, from the one or more Laplacian electrodes, and from the one or more pressure sensors.

2. A method comprising
receiving athlete data including motion data from one or more inertial measurement units associated with one or more muscles of an athlete engaging in athlete activity,
using the motion data to characterize the athlete activity or a status of the athlete,
receiving from one or more temperature sensors integrated with the initial measurement units athlete data comprising one or more body temperatures of the athlete, and
determining one or more athlete performance factors based on the one or more body temperatures of the athlete.

3. A method comprising
receiving athlete data from concentric electrodes in contact with skin of an athlete at one or more innervation zones of a muscle group, and
determining electromyography information about the muscle group based on signal morphology of the received athlete data.

4.-24. (canceled)
